SEQUENCE LISTING (49 sequences)

SEQ ID NO: 1 - DNA, 879 bp, Artificial Sequence
  Synthetic
atgtccctgtttccatcactccctctccttctcctgagtatggtggcagcgtcttactcagaaactgaacagaaactgatctctgaagaggacctgcaccatcatcaccaccattcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctggatccccaggtccgaagggccaaaaaggagacccgggaaaaagtccggatggtgatagtagcctggctgcctcagaacgcaaagctcttcagacagaaatggcacgtatcaaaaagtggctgacccaactcaaggcggctgttgaacggctttgccatccttgcccgtgggaatggacttttttccagggtaactgttatttcatgagcaattcccagcgtaactggcacgactccatcactgcttgcaaagaagtgggtgcgcagctcgtcgtgatcaaatccgcggaagaacagaattttctgcagctccaatccagccgtagcaatcggttcacctggatgggtctttctgaccttaaccaggagggtacctggcaatgggtcgatggatcccctctcctcccttccttcaaacagtattggaaccgcggtgaacctaacaacgtcggcgaggaggattgcgcagagttcagcggtaacggctggaatgacgacaagtgcaatctcgctaagttctggatctgcaaaaagtccgctgcgtcttgcagccgggacgaggaacag

SEQ ID NO: 2 - protein, 293 aa, Artificial Sequence
  Synthetic
MSLFPSLPLLLLSMVAASYSETEQKLISEEDLHHHHHHSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQ

SEQ ID NO: 3 - DNA, 882 bp, Artificial Sequence
  Synthetic
atgtccctgtttccatcactccctctccttctcctgagtatggtggcagcgtcttactcagaaactgaacagaaactgatctctgaagaggacctgcaccatcatcaccaccattcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctggatccccaggtccgaagggccaaaaaggagacccgggaaaaagtccggatggtgatagtagcctggctgcctcagaacgcaaagctcttcagacagaaatggcacgtatcaaaaagtggctgaccactcaactcaaggcggctgttgaacggctttgccatccttgcccgtgggaatggacttttttccagggtaactgttatttcatgagcaattcccagcgtaactggcacgactccatcactgcttgcaaagaagtgggtgcgcagctcgtcgtgatcaaatccgcggaagaacagaattttctgcagctccaatccagccgtagcaatcggttcacctggatgggtctttctgaccttaaccaggagggtacctggcaatgggtcgatggatcccctctcctcccttccttcaaacagtattggaaccgcggtgaacctaacaacgtcggcgaggaggattgcgcagagttcagcggtaacggctggaatgacgacaagtgcaatctcgctaagttctggatctgcaaaaagtccgctgcgtcttgcagccgggacgaggaacag

SEQ ID NO: 4 - protein, 294 aa, Artificial Sequence
  Synthetic
MSLFPSLPLLLLSMVAASYSETEQKLISEEDLHHHHHHSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQ

SEQ ID NO: 5 - DNA, 885 bp, Artificial Sequence
  Synthetic
atgtccctgtttccatcactccctctccttctcctgagtatggtggcagcgtcttactcagaaactgaacagaaactgatctctgaagaggacctgcaccatcatcaccaccattcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctggatccccaggtccgaagggccaaaaaggagacccgggaaaaagtccggatggtgatagtagcctggctgcctcagaacgcaaagctcttcagacagaaatggcacgtatcaaaaagtggctgaccctgactcaactcaaggcggctgttgaacggctttgccatccttgcccgtgggaatggacttttttccagggtaactgttatttcatgagcaattcccagcgtaactggcacgactccatcactgcttgcaaagaagtgggtgcgcagctcgtcgtgatcaaatccgcggaagaacagaattttctgcagctccaatccagccgtagcaatcggttcacctggatgggtctttctgaccttaaccaggagggtacctggcaatgggtcgatggatcccctctcctcccttccttcaaacagtattggaaccgcggtgaacctaacaacgtcggcgaggaggattgcgcagagttcagcggtaacggctggaatgacgacaagtgcaatctcgctaagttctggatctgcaaaaagtccgctgcgtcttgcagccgggacgaggaacag

SEQ ID NO: 6 - protein, 295 aa, Artificial Sequence
  Synthetic
MSLFPSLPLLLLSMVAASYSETEQKLISEEDLHHHHHHSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTLTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQ

SEQ ID NO: 7 - DNA, 921 bp, Artificial Sequence
  Synthetic
atgtccctgtttccatcactccctctccttctcctgagtatggtggcagcgtcttactcagaaactgaacagaaactgatctctgaagaggacctgcaccatcatcaccaccattcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctggatccccaggtccgaagggccaaaaaggagacccgggaaaaagtccggatggtgatagtagcctggctgcctcagaacgcaaagctcttcagacagaaatggcacgtatcaaaaagtggctgacccaactcaaggcggctgttgaacggctttgccatccttgcccgtgggaatggacttttttccagggtaactgttatttcatgagcaattcccagcgtaactggcacgactccatcactgcttgcaaagaagtgggtgcgcagctcgtcgtgatcaaatccgcggaagaacagaattttctgcagctccaatccagccgtagcaatcggttcacctggatgggtctttctgaccttaaccaggagggtacctggcaatgggtcgatggatcccctctcctcccttccttcaaacagtattggaaccgcggtgaacctaacaacgtcggcgaggaggattgcgcagagttcagcggtaacggctggaatgacgacaagtgcaatctcgctaagttctggatctgcaaaaagtccgctgcgtcttgcagccgggacgaggaacagttcctctcccctgcgcctgctacccctaatcctccccctgcg

SEQ ID NO: 8 - protein, 307 aa, Artificial Sequence
  Synthetic
MSLFPSLPLLLLSMVAASYSETEQKLISEEDLHHHHHHSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 9 - DNA, 924 bp, Artificial Sequence
  Synthetic
atgtccctgtttccatcactccctctccttctcctgagtatggtggcagcgtcttactcagaaactgaacagaaactgatctctgaagaggacctgcaccatcatcaccaccattcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctggatccccaggtccgaagggccaaaaaggagacccgggaaaaagtccggatggtgatagtagcctggctgcctcagaacgcaaagctcttcagacagaaatggcacgtatcaaaaagtggctgaccactcaactcaaggcggctgttgaacggctttgccatccttgcccgtgggaatggacttttttccagggtaactgttatttcatgagcaattcccagcgtaactggcacgactccatcactgcttgcaaagaagtgggtgcgcagctcgtcgtgatcaaatccgcggaagaacagaattttctgcagctccaatccagccgtagcaatcggttcacctggatgggtctttctgaccttaaccaggagggtacctggcaatgggtcgatggatcccctctcctcccttccttcaaacagtattggaaccgcggtgaacctaacaacgtcggcgaggaggattgcgcagagttcagcggtaacggctggaatgacgacaagtgcaatctcgctaagttctggatctgcaaaaagtccgctgcgtcttgcagccgggacgaggaacagttcctctcccctgcgcctgctacccctaatcctccccctgcg

SEQ ID NO: 10 - protein, 308 aa, Artificial Sequence
  Synthetic
MSLFPSLPLLLLSMVAASYSETEQKLISEEDLHHHHHHSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 11 - DNA, 927 bp, Artificial Sequence
  Synthetic
atgtccctgtttccatcactccctctccttctcctgagtatggtggcagcgtcttactcagaaactgaacagaaactgatctctgaagaggacctgcaccatcatcaccaccattcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctggatccccaggtccgaagggccaaaaaggagacccgggaaaaagtccggatggtgatagtagcctggctgcctcagaacgcaaagctcttcagacagaaatggcacgtatcaaaaagtggctgaccctgactcaactcaaggcggctgttgaacggctttgccatccttgcccgtgggaatggacttttttccagggtaactgttatttcatgagcaattcccagcgtaactggcacgactccatcactgcttgcaaagaagtgggtgcgcagctcgtcgtgatcaaatccgcggaagaacagaattttctgcagctccaatccagccgtagcaatcggttcacctggatgggtctttctgaccttaaccaggagggtacctggcaatgggtcgatggatcccctctcctcccttccttcaaacagtattggaaccgcggtgaacctaacaacgtcggcgaggaggattgcgcagagttcagcggtaacggctggaatgacgacaagtgcaatctcgctaagttctggatctgcaaaaagtccgctgcgtcttgcagccgggacgaggaacagttcctctcccctgcgcctgctacccctaatcctccccctgcg

SEQ ID NO: 12 - protein, 309 aa, Artificial Sequence
  Synthetic
MSLFPSLPLLLLSMVAASYSETEQKLISEEDLHHHHHHSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTLTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 13 - DNA, 816 bp, Artificial Sequence
  Synthetic
atgtccctgtttccatcactccctctccttctcctgagtatggtggcagcgtcttactcagaaactgaacagaaactgatctctgaagaggacctgcaccatcatcaccaccattcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctggatccccaggtccgaagggccaaaaaggagacccgggaaaaagtccggatggtgatagtagcctggctgcctcagaacgcaaagctcttcagacagaaatggcacgtatcaaacatccttgcccgtgggaatggacttttttccagggtaactgttatttcatgagcaattcccagcgtaactggcacgactccatcactgcttgcaaagaagtgggtgcgcagctcgtcgtgatcaaatccgcggaagaacagaattttctgcagctccaatccagccgtagcaatcggttcacctggatgggtctttctgaccttaaccaggagggtacctggcaatgggtcgatggatcccctctcctcccttccttcaaacagtattggaaccgcggtgaacctaacaacgtcggcgaggaggattgcgcagagttcagcggtaacggctggaatgacgacaagtgcaatctcgctaagttctggatctgcaaaaagtccgctgcgtct

SEQ ID NO: 14 - protein, 272 aa, Artificial Sequence
  Synthetic
MSLFPSLPLLLLSMVAASYSETEQKLISEEDLHHHHHHSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAAS

SEQ ID NO: 15 - DNA, 819 bp, Artificial Sequence
  Synthetic
atgtccctgtttccatcactccctctccttctcctgagtatggtggcagcgtcttactcagaaactgaacagaaactgatctctgaagaggacctgcaccatcatcaccaccattcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctggatccccaggtccgaagggccaaaaaggagacccgggaaaaagtccggatggtgatagtagcctggctgcctcagaacgcaaagctcttcagacagaaatggcacgtatcaaaaagcatccttgcccgtgggaatggacttttttccagggtaactgttatttcatgagcaattcccagcgtaactggcacgactccatcactgcttgcaaagaagtgggtgcgcagctcgtcgtgatcaaatccgcggaagaacagaattttctgcagctccaatccagccgtagcaatcggttcacctggatgggtctttctgaccttaaccaggagggtacctggcaatgggtcgatggatcccctctcctcccttccttcaaacagtattggaaccgcggtgaacctaacaacgtcggcgaggaggattgcgcagagttcagcggtaacggctggaatgacgacaagtgcaatctcgctaagttctggatctgcaaaaagtccgctgcgtct

SEQ ID NO: 16 - protein, 273 aa, Artificial Sequence
  Synthetic
MSLFPSLPLLLLSMVAASYSETEQKLISEEDLHHHHHHSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAAS

SEQ ID NO: 17 - DNA, 822 bp, Artificial Sequence
  Synthetic
atgtccctgtttccatcactccctctccttctcctgagtatggtggcagcgtcttactcagaaactgaacagaaactgatctctgaagaggacctgcaccatcatcaccaccattcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctggatccccaggtccgaagggccaaaaaggagacccgggaaaaagtccggatggtgatagtagcctggctgcctcagaacgcaaagctcttcagacagaaatggcacgtatcaaaaagtggcatccttgcccgtgggaatggacttttttccagggtaactgttatttcatgagcaattcccagcgtaactggcacgactccatcactgcttgcaaagaagtgggtgcgcagctcgtcgtgatcaaatccgcggaagaacagaattttctgcagctccaatccagccgtagcaatcggttcacctggatgggtctttctgaccttaaccaggagggtacctggcaatgggtcgatggatcccctctcctcccttccttcaaacagtattggaaccgcggtgaacctaacaacgtcggcgaggaggattgcgcagagttcagcggtaacggctggaatgacgacaagtgcaatctcgctaagttctggatctgcaaaaagtccgctgcgtct

SEQ ID NO: 18 - protein, 274 aa, Artificial Sequence
  Synthetic
MSLFPSLPLLLLSMVAASYSETEQKLISEEDLHHHHHHSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAAS

SEQ ID NO: 19 - DNA, 930 bp, Artificial Sequence
  Synthetic
atgtccctgtttccatcactccctctccttctcctgagtatggtggcagcgtcttactcagaaactgaacagaaactgatctctgaagaggacctgcaccatcatcaccaccattcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctggatccccaggtccgaagggccaaaaaggagacccgggaaaaagtccggatggtgatagtagcctggctgcctcagaacgcaaagctcttcagacagaaatggcacgtatcaaaaagtggctgacctctggcactcaactcaaggcggctgttgaacggctttgccatccttgcccgtgggaatggacttttttccagggtaactgttatttcatgagcaattcccagcgtaactggcacgactccatcactgcttgcaaagaagtgggtgcgcagctcgtcgtgatcaaatccgcggaagaacagaattttctgcagctccaatccagccgtagcaatcggttcacctggatgggtctttctgaccttaaccaggagggtacctggcaatgggtcgatggatcccctctcctcccttccttcaaacagtattggaaccgcggtgaacctaacaacgtcggcgaggaggattgcgcagagttcagcggtaacggctggaatgacgacaagtgcaatctcgctaagttctggatctgcaaaaagtccgctgcgtcttgcagccgggacgaggaacagttcctctcccctgcgcctgctacccctaatcctccccctgcg

SEQ ID NO: 20 - protein, 310 aa, Artificial Sequence
  Synthetic
MSLFPSLPLLLLSMVAASYSETEQKLISEEDLHHHHHHSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTSGTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 21 - DNA, 936 bp, Artificial Sequence
  Synthetic
atgtccctgtttccatcactccctctccttctcctgagtatggtggcagcgtcttactcagaaactgaacagaaactgatctctgaagaggacctgcaccatcatcaccaccattcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctggatccccaggtccgaagggccaaaaaggagacccgggaaaaagtccggatggtgatagtagcctggctgcctcagaacgcaaagctcttcagacagaaatggcacgtatcaaaaagtggctgacctctggcgggtccactcaactcaaggcggctgttgaacggctttgccatccttgcccgtgggaatggacttttttccagggtaactgttatttcatgagcaattcccagcgtaactggcacgactccatcactgcttgcaaagaagtgggtgcgcagctcgtcgtgatcaaatccgcggaagaacagaattttctgcagctccaatccagccgtagcaatcggttcacctggatgggtctttctgaccttaaccaggagggtacctggcaatgggtcgatggatcccctctcctcccttccttcaaacagtattggaaccgcggtgaacctaacaacgtcggcgaggaggattgcgcagagttcagcggtaacggctggaatgacgacaagtgcaatctcgctaagttctggatctgcaaaaagtccgctgcgtcttgcagccgggacgaggaacagttcctctcccctgcgcctgctacccctaatcctccccctgcg

SEQ ID NO: 22 - protein, 312 aa, Artificial Sequence
  Synthetic
MSLFPSLPLLLLSMVAASYSETEQKLISEEDLHHHHHHSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTSGGSTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 23 - DNA, 942 bp, Artificial Sequence
  Synthetic
atgtccctgtttccatcactccctctccttctcctgagtatggtggcagcgtcttactcagaaactgaacagaaactgatctctgaagaggacctgcaccatcatcaccaccattcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctggatccccaggtccgaagggccaaaaaggagacccgggaaaaagtccggatggtgatagtagcctggctgcctcagaacgcaaagctcttcagacagaaatggcacgtatcaaaaagtggctgacctctggcggaggcgggtccactcaactcaaggcggctgttgaacggctttgccatccttgcccgtgggaatggacttttttccagggtaactgttatttcatgagcaattcccagcgtaactggcacgactccatcactgcttgcaaagaagtgggtgcgcagctcgtcgtgatcaaatccgcggaagaacagaattttctgcagctccaatccagccgtagcaatcggttcacctggatgggtctttctgaccttaaccaggagggtacctggcaatgggtcgatggatcccctctcctcccttccttcaaacagtattggaaccgcggtgaacctaacaacgtcggcgaggaggattgcgcagagttcagcggtaacggctggaatgacgacaagtgcaatctcgctaagttctggatctgcaaaaagtccgctgcgtcttgcagccgggacgaggaacagttcctctcccctgcgcctgctacccctaatcctccccctgcg

SEQ ID NO: 24 - protein, 314 aa, Artificial Sequence
  Synthetic
MSLFPSLPLLLLSMVAASYSETEQKLISEEDLHHHHHHSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTSGGGGSTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 25 - protein, 822 aa, Artificial Sequence
  Synthetic
ATGTCCCTGTTTCCATCACTCCCTCTCCTTCTCCTGAGTATGGTGGCAGCGTCTTACTCAGAAACTGTGACCTGTGAGGATGCCCAAAAGACCTGCCCTGCAGTGATTGCCTGTAGCTCTCCAGGCATCAACGGCTTCCCAGGCAAAGATGGGCGTGATGGCACCAAGGGAGAAAAGGGGGAACCAGGCCAAGGGCTCAGAGGCTTACAGGGCCCCCCTGGAAAGTTGGGGCCTCCAGGAAATCCAGGGCCTTCTGGATCCCCAGGTCCGAAGGGCCAAAAAGGAGACCCGGGAAAAAGTCCGGATGGTGATAGTAGCCTGGCTGCCTCAGAACGCAAAGCTCTTCAGACAGAAATGGCACGTATCAAAAAGTGGCTGACCCAACTCAAGGCGGCTGTTGAACGGCTTTGCCATCCTTGCCCGTGGGAATGGACTTTTTTCCAGGGTAACTGTTATTTCATGAGCAATTCCCAGCGTAACTGGCACGACTCCATCACTGCTTGCAAAGAAGTGGGTGCGCAGCTCGTCGTGATCAAATCCGCGGAAGAACAGAATTTTCTGCAGCTCCAATCCAGCCGTAGCAATCGGTTCACCTGGATGGGTCTTTCTGACCTTAACCAGGAGGGTACCTGGCAATGGGTCGATGGATCCCCTCTCCTCCCTTCCTTCAAACAGTATTGGAACCGCGGTGAACCTAACAACGTCGGCGAGGAGGATTGCGCAGAGTTCAGCGGTAACGGCTGGAATGACGACAAGTGCAATCTCGCTAAGTTCTGGATCTGCAAAAAGTCCGCTGCGTCTTGCAGCCGGGACGAGGAACAG

SEQ ID NO: 26 - protein, 274 aa, Artificial Sequence
  Synthetic
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQ

SEQ ID NO: 27 - DNA, 864 bp, Artificial Sequence
  Synthetic
atgtccctgtttccatcactccctctccttctcctgagtatggtggcagcgtcttactcagaaactgtgacctgtgaggatgcccaaaagacctgccctgcagtgattgcctgtagctctccaggcatcaacggcttcccaggcaaagatgggcgtgatggcaccaagggagaaaagggggaaccaggccaagggctcagaggcttacagggcccccctggaaagttggggcctccaggaaatccagggccttctggatccccaggtccgaagggccaaaaaggagacccgggaaaaagtccggatggtgatagtagcctggctgcctcagaacgcaaagctcttcagacagaaatggcacgtatcaaaaagtggctgacccaactcaaggcggctgttgaacggctttgccatccttgcccgtgggaatggacttttttccagggtaactgttatttcatgagcaattcccagcgtaactggcacgactccatcactgcttgcaaagaagtgggtgcgcagctcgtcgtgatcaaatccgcggaagaacagaattttctgcagctccaatccagccgtagcaatcggttcacctggatgggtctttctgaccttaaccaggagggtacctggcaatgggtcgatggatcccctctcctcccttccttcaaacagtattggaaccgcggtgaacctaacaacgtcggcgaggaggattgcgcagagttcagcggtaacggctggaatgacgacaagtgcaatctcgctaagttctggatctgcaaaaagtccgctgcgtcttgcagccgggacgaggaacagttcctctcccctgcgcctgctacccctaatcctccccctgcg

SEQ ID NO: 28 - protein, 288 aa, Artificial Sequence
  Synthetic
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 29 - protein, 21 aa, Artifical Sequence
  Synthetic
GGTCAGCCACTTTTTGATACG

SEQ ID NO: 30 - protein, 40 aa, Artifical Sequence
  Synthetic
CGTATCAAAAAGTGGCTGACCCAACTCAAGGCGGCTGTTG

SEQ ID NO: 31 - protein, 40 aa, Artifical Sequence
  Synthetic
CGTATCAAAAAGTGGCTGACCACTCAACTCAAGGCGGCTG

SEQ ID NO: 32 - protein, 40 aa, Artifical sequence
  Synthetic
CGTATCAAAAAGTGGCTGACCCTGACTCAACTCAAGGCGG

SEQ ID NO: 33 - protein, 20 aa, Artifical sequence
  Synthetic
CATCCTTGCCCGTGGGAATG

SEQ ID NO: 34 - protein, 42 aa, Artifical sequence
  Synthetic
CATTCCCACGGGCAAGGATGTTTGATACGTGCCATTTCTGTC

SEQ ID NO: 35 - protein, 43 aa, Artifical sequence
  Synthetic
CATTCCCACGGGCAAGGATGCTTTTTGATACGTGCCATTTCTG

SEQ ID NO: 36 - protein, 44 aa, Artifical sequence
  Synthetic
CATTCCCACGGGCAAGGATGCCACTTTTTGATACGTGCCATTTC

SEQ ID NO: 37 - protein, 45 aa, Artifical sequence
  Synthetic
GCCCGCGAATTCGCAAGCTTTATTACTGTTCCTCGTCCCGGCAAG

SEQ ID NO: 38 - protein, 248 aa, Homo Sapiens
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 39 - protein, 53 aa, Homo sapiens
GINGFGKDGRDGTKGEKGEPGQGLRGLQGPGKLGPGNGPSGSGPKGQKGDGKS

SEQ ID NO: 40 - protein, 50 aa, Rattus norvegicus
GRDGRDGPKGEKGEPGQGLRGLQGPGKLGPGSVGAGSQGPKGQKGDRGDS

SEQ ID NO: 41 - protein, 55 aa, Rattus norvegicus
GLNGFGKDGHDGAKGEKGEPGQGLRGLQGPGKVGPAGPGNSGKGATGPKGDRGES

SEQ ID NO: 42 - protein, 49 aa, Mus musculus
GRDGRDGPKGEKGEPGQGLRGLQGPGKLGPGSVGSGSGPKGQKGDHGDN

SEQ ID NO: 43 - protein, 55 aa, Mus musculus
GLNGFGKDGRDGAKGEKGEPGQGLRGLQGPGKVGPTGPGNGLKGAVGPKGDRGDR

SEQ ID NO: 44 - protein, 53 aa, Macaca mulatta
GINGFGKDGRDGTKGEKGEPGQGLRGLQGPGKLGPGNGSSGSGPKGQKGDGES

SEQ ID NO: 45 - protein, 15 aa, Homo sapiens
  misc_feature (2)..(2): Xaa can be any naturally occurring amino acid
GXYGXYGXGKYGPYG

SEQ ID NO: 46 - protein, 181 aa, Artifical sequence
  Synthetic
EPPTQKPKKIVNAKKDVVNTKMFEELKSRLDTLAQEVALLKEQQALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 47 - protein, 36 aa, Artifical sequence
  Synthetic
XXXXXXXXXLXXXXXXLXXEVXXLKEXQALQTVCLX

SEQ ID NO: 48 - protein, 113 aa, Artifical sequence
  Synthetic
ETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQ

SEQ ID NO: 49 - protein, 16 aa, Artifical sequence
  Synthetic
GLRGLQGPPGKLGPPG

(19) United States
(12) Patent Application Publication
(10) Pub. No.: US 2011/0027267 A1
(43) Pub. Date: Feb. 3, 2011

(54) Fusion Proteins of Mannose Binding Lectins for Treatment of Disease

(75) Inventors: Majbritt Hauge Kyneb, Viby J. (DK); Mikkel Holmen Andersen, Sporup (DK); Michael Etzerodt, Hinnerup (DK); Thor Las Holtet, Ronde (DK)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: ANAPHORE, INC., LA JOLLA, CA (US)

(21) Appl. No.: 12/741,702

(22) PCT Filed: Nov. 10, 2008
(86) PCT No.: PCT/US08/83062

§ 371 Date: May 6, 2010

Related U.S. Application Data
(60) Provisional application No. 60/996,288, filed on Nov. 9, 2007.

(51) Int. Cl. A61K 39/395; C07K 14/00; A61K 38/16; A61K 39/00; C07H 21/00; C12N 15/63; C12N 5/10; C12P 21/02; A61P 35/00

(52) U.S. Cl. 424/133.1; 530/350; 514/21.2; 424/130.1; 424/185.1; 536/23.1; 435/320.1; 435/325; 435/69.7

(57) ABSTRACT

Fusion proteins having sequences that target specific moieties such as carbohydrates, lipids, and/or proteins that are associated with certain cell types and/or pathogens; and a sequence that induces effector function are provided. The disclosure also provides nucleic acids encoding the fusion proteins, as well as pharmaceutical compositions, methods of use, and methods of treating conditions or diseases such as infectious diseases, cancers, immune related disorders and other ailments, that include the fusions proteins described herein.

### CROSS-REFERENCE TO RELATED APPLICATIONS

This application claims the benefit of U.S. Provisional Patent Application Ser. No. 60/996,288, filed Nov. 9, 2007, and is incorporated herein by reference.

### FIELD OF THE INVENTION

The present invention relates to the treatment of various diseases and infections. More particularly, the invention relates to fusion proteins including a mannose binding lectin polypeptide sequence. The fusion proteins can be used in a pharmaceutical composition for treating infectious diseases, cancers, immune related disorders and other ailments.

### BACKGROUND OF THE INVENTION

Mannose-binding lectin (MBL), also called mannose binding protein (MBP), is a calcium-dependent serum protein that plays a role in the innate immune response by binding to carbohydrates on the surface of a wide range of pathogens (viruses, bacteria, fungi, protozoa) where it can activate the complement system. MBL serves also as a direct opsonin and mediates binding and uptake of pathogens by tagging the surface of a pathogen to facilitate recognition and ingestion by phagocytes.

Mannose-binding lectin is a member of the collectin family of proteins, which are made in the liver. Collectins get their name because they have a collagen-like region and a lectin region. Lectins are proteins that bind carbohydrates, usually on the surface of bacteria. The collagen domain interacts with the effector parts of the innate immune system. The MBL2 gene on human chromosome 10 produces MBL, an oligomer of 248-amino acid protein subunits composed of three identical polypeptide chains comprising a N-terminal cysteine rich region, a collagen-like region, a neck region, and a carbohydrate recognition domain (CRD). Three MBL polypeptide chains assemble into a biologically active trimer found in vivo.

When serum MBL interacts with carbohydrates on the surface of microorganisms, it forms the pathogen recognition component of the lectin pathway of complement activation. MBL binds to surface arrays containing repeated mannose or N-acetylglucosamine residues. It circulates as a complex with one or more MBP-associated serine proteases (MASPs) that autoactivate when the complex binds to an appropriate surface.

The surface recognition function of MBP is mediated by clusters of three C-type carbohydrate-recognition domains (CRDs) held together by coiled-coils of α-helices. The N-terminal portion collagen-like domain is composed of Gly-X-Y triplets with a single interruption that forms a bend in the domain. The short N-terminal domain contains several cysteine residues that form interchain disulfide bonds. Serum MBLs assemble into larger forms containing 2-4 trimeric subunits in rodents and as many as six subunits in humans. All three oligomeric forms of rat serum MBP, designated MBP-A, can fix complement, although the larger oligomers have higher specific activity. Many species express a second form of MBP. In rats, the second form, MBP-C, is found in the liver. MBP-C does not form higher oligomers beyond the simple subunit that contains three polypeptides. Analysis of chimeras between rat MBP-A and MBP-C suggests that the collagen-like domains contain the MASP-binding sites.

MBL has been studied as a therapeutic for several years. For example, MBL has been considered as a treatment of infections for individuals treated with tumor necrosis factor (TNF) inhibitors resulting in impaired phagocytic function (see WO02/05833). MBL is used in its natural form to allow for clearance of infections through binding of the MBL CRD region to the infectious agents and subsequent clearance, thereby compensating for the lack of phagocytic function in a subset of patients treated with TNF inhibitors. MBL has also been considered in fusion proteins with TNF superfamily ligands for use as vaccine adjuvant (see U.S. Pat. No. 7,300,744). The previously described use of collectins such as MBL enabled production of a trimeric TNF-based family of molecules such as CD40L that activates dendritic cells and T cells to mount an immune response. The higher order multimerization as described in the above applications is desirable for adjuvant properties. Thus, these methods provide a molecule that may activate innate and adaptive immune functions to provide an antibody response. However, these methods are not designed to initiate complement activation. In fact, complement activation would be detrimental to a vaccine effect since it would result in killing of desired immune cells to which the MBL fusion protein has bound. In the present application, MBP is used to mediate complement activation, whereas triggering a cellular immune response and antibody production is highly undesirable.

Accordingly, the inventors have identified a need in the art to provide a method of treating infection, cancers, and other disorders activating complement.

### SUMMARY OF THE INVENTION

In an aspect, the invention provides a fusion protein comprising a first polypeptide comprising a mannose binding lectin (MBL) polypeptide having effector function and a second polypeptide comprising a targeting sequence that binds to a cell surface or to a virus, wherein the first polypeptide does not comprise an active MBL C-Type Lectin Like Domain (CLTD). The targeting sequence of the second polypeptide can bind to a targeted moiety on the surface of a cell selected from the group consisting of tumor cells, immune cells, bacterial cells, protozoa, fungi and a cell infected with a virus. The targeted moiety can comprise any one or combination of carbohydrate, lipid, or amino acid sequence that is associated with a particular cell. The targeting sequence can comprise a lectin, including a C-type lectin domain (CTLD). The first polypeptide binds comprises a sequence that allows for effector function, such as inducing a mammalian complement system.

In an aspect, the invention provides a method of activating a mammalian complement system comprising administering to the mammal a fusion protein comprising a mannose binding lectin (MBL) polypeptide having effector function and a second polypeptide comprising a targeting sequence that binds to a cell surface or to a virus, wherein the first polypeptide does not comprise an active MBL C-Type Lectin Like Domain (CLTD).

In an aspect, the invention provides a pharmaceutical composition comprising the fusion protein of the invention and a pharmaceutically acceptable excipient. The pharmaceutical composition of the invention can further comprising at least one additional therapeutic agent, such as a chemotherapeutic agent and/or a targeted therapeutic agent, such as an antibody, a kinase inhibitor, or a cancer vaccine.

In another aspect the invention provides a method of treating a pathogenic disease comprising administering to a patient suffering from the disease and effective amount of the fusion protein of the invention, or a pharmaceutical composition thereof, wherein the targeting sequence binds to a cell surface marker of the pathogen or a marker on a cell that is infected with a virus.

In another aspect, the invention provides a method of treating a proliferative disease associated with tumor cells comprising administering to a patient in need thereof an effective amount of the fusion protein of the invention, or a pharmaceutical composition thereof, wherein the targeting sequence binds to a marker on the surface of the tumor cells, such as on the surface of a cancer cell.

In other aspects, the invention relates to isolated nucleic acids comprising a sequence encoding a fusion protein of the invention, expression vectors comprising the nucleic acids, host cells comprising the expression vectors, and methods for the preparation of the fusion protein of the invention comprising the nucleic acids, vectors, and host cells.

Other aspects of the invention will be apparent to those of skill in the art from the following detailed description of the invention.

### BRIEF DESCRIPTION OF THE FIGURES

FIG. 1 depicts the polypeptide sequence of full length human MBP (SEQ ID NO: 38) and the general structural regions (signal peptide region, multimerizing region, collagen-like region, coiled coil region, and C-type lectin domain). The italicized and underlined amino acids are thought to include the binding region for MBP associated serine proteases (MASPs).

FIG. 2 illustrates an alignment of various MBP sequences from human (SEQ ID NO: 39), rat (MBP-A is SEQ ID NO: 40; MBP-C is SEQ ID NO: 41), mouse (MBP-A is SEQ ID NO: 42; MBP-C is SEQ ID NO: 43), and monkey (SEQ ID NO: 43). The residue “O” stands for hydroxyproline; asterisks (*) denote amino acid residues that are conserved across MBPs and ficolins. Bolded, underlined amino acid residues are identified as important in MBP interaction with MBP-associated serine protease (MASP) [See, Wallis, et al., J. Biol. Chem., 279(14):14065-073 (2004)]. One embodiment of functional variants of the MASP binding region of human MBP are defined in SEQ ID NO: 45.

FIG. 3 shows ELISA binding analyses of the tagged MBP/DC-SIGN CTLD fusion proteins binding to immobilised Le^{y} HSA. (A) Binding activity of various MBP/DC-SIGN constructs at 4 days post transfection. ACsC shows strongest binding activity. (B) Comparative binding of DC-SIGN/Fc and MBP/DC-SIGN (ACsC), each with and without various competitors. (C) Additional binding activity assays of various MBP/DC-SIGN constructs at 4 days post transfection.

FIG. 4 shows binding of MBP/DC-SIGN CTLD fusion protein binding to SKBR-3 cells using suspension phase ELISA. (A) Constructs Abs and ABsC demonstrate higher binding activity than the positive control DC-SIGN/Fc. (B) Binding specificity of MBP/DC-SIGN ABs construct for Le^{y} in the presence of various competitors and calcium. (C) ELISA of MBP/DC-SIGN constructs Abs and ABsC to MCF-7 cells as compared to positive and negative controls.

FIG. 5 shows elution profiles of the MBP/DC-Sign CTLD ABs and −ABsC on a 25 mL mannan-agarose column (Sigma).

FIG. 6 shows SDS-PAGE analysis of the isolated MBP/DC-Sign CTLD-ABS (A), and MBP/DC-Sign CTLD-AbsC (B) derivatives. The left-most lane in the gels are molecular weight standard size ladders.

FIG. 7 shows Western blot analysis (non-reduced) of the oligomerisation profile of MBP/DC-SIGN CTLD ABs isolated by mannan-sepharose affinity purification. The blot shows that the majority of the purified ABs construct is present as higher order oligomers.

FIG. 8 shows ELISA binding results for MBP/DC-SIGN CTLD ABs (▪) and DC-SIGN-Fc (♦) binding to immobilized Lewis Y-HSA. The multimerizing domain of MBP provides for increased avidity gain relative to the DC-SIGN-Fc molecule.

FIG. 9 shows induction of C4 complement activation by MBP/DC-SIGN CTLD ABs—with MASP dependant cleavage of C4.

FIG. 10 shows MBP/DC-SIGN CTLD ABs (-♦-)—MASP dependant conversion of C4 on SKBR-3 cells.

FIG. 11 shows analysis of inhibition of SKBR-3 (A) and MCF-7 (B) cell proliferation by MBP/DC-SIGN CTLD derivatives ABsC and −ABsC0, or Herceptin. Graph legend: diamond (-♦-) MBP/DC-SIGN; Square (-▪-) MBP-DC-SIGN-ABsC0+5 μg/mL herceptin; Triangle (-▴-) Herceptin; “X” (-x-) TBSC buffer; asterisk (-*-) Medium only.

### DETAILED DESCRIPTION OF THE INVENTION

The present invention takes advantage of the complement fixation activity of MBP by constructing a series of fusion proteins comprising a first polypeptide comprising a mannose binding lectin (MBL) polypeptide that can induce complement fixation, and second polypeptide that comprises a sequence that target moieties that are associated with particular cell types and pathogens. In one aspect, the invention provides a fusion protein comprising a first polypeptide comprising a mannose binding lectin polypeptide, wherein the first polypeptide has effector function; and a second polypeptide comprising a sequence that binds to a targeted moiety.

In another aspect, the invention is directed to the treatment of disease by providing to a subject in need a fusion protein that includes the effector function of MBL and a targeting sequence that directs the fusion protein to a cell or other pathogen of interest. Once associated with the cell or pathogen, the effector function of the MBL activates the complement system of the host, thereby initiating opsonization and ultimately phagocytosis of the cell or pathogen. The fusion protein of the invention lacks the MBL carbohydrate recognition domain, or an active MBP C-Type Lectin-Like Domain, such that the MBL fusion protein, while retaining effector function, does not have an MBL sequence that would otherwise binds to mannose or other oligosaccharides on cell surfaces.

### DEFINITIONS

Before defining the invention in further detail, a number of terms are defined. Unless a particular definition for a term is provided herein, the terms and phrases used throughout this disclosure should be taken to have the meaning as commonly understood in the art. Also, as used in this specification and the appended claims, the singular forms “a,” “an,” and “the” include plural referents unless the context clearly dictates otherwise.

As used herein, “effector function” means the ability to induce an immune response in a mammal that is not associated with an antibody or T cell response. For example effector function of mannose binding protein (MBP) activates the complement system, which is a set of plasma proteins that work together to attack extracellular pathogens. While the most important role of the complement system is opsonization (coating foreign organisms with a receptor recognized by phagocytes), it also recruits inflammatory cells and kills pathogens directly through membrane attack complexes. In mammals, activating or “fixing” complement generally means that MBP binds to the serum proteins C1, C2, C3, C4, C5, C6, C7, C8, and C9, collectively called “complement,” and thereby stimulate the binding of macrophages to the protein and facilitate subsequent ingestion by those macrophages.

A “tetranectin trimerizing domain” refers to a trimerizing domain derived from tetranectin as described in U.S. Patent Application Publication No. 2007/0154901 ('901 Application), which is incorporated by reference in its entirety. The mature human tetranectin single chain polypeptide sequence is provided herein as SEQ ID NO: 46. Examples of a tetranectin trimerizing domain includes the amino acids 17 to 49, 17 to 50, 17 to 51 and 17-52 of SEQ ID NO: 46, which represent the amino acids encoded by exon 2 of the human tetranectin gene, and optionally the first one, two or three amino acids encoded by exon 3 of the gene. Other examples include amino acids 1 to 49, 1 to 50, 1 to 51 and 1 to 52, which represents all of exons 1 and 2, and optionally the first one, two or three amino acids encoded by exon 3 of the gene. Alternatively, only a part of the amino acid sequence encoded by exon 1 is included in the trimerizing domain. In particular, the N-terminus of the trimerizing domain may begin at any of residues 1, 2, 3, 4, 5, 6, 7, 8, 9, 10, 11, 12, 13, 14, 15, 16 and 17 of SEQ ID NO: 46. In particular embodiments, the N terminus is 110 or V17 and the C-terminus is Q47, T48, V49, C(S)50, L51 or K52 (numbering according to SEQ ID NO: 46).

In one aspect of the invention, the trimerizing domain is a tetranectin trimerizing structural element (“TTSE”) having an amino acid sequence of SEQ ID NO: 47 which is a consensus sequence of the tetranectin family trimerizing structural element as more fully described in US 2007/00154901. The TTSE embraces variants of a naturally occurring member of the tetranectin family of proteins, and in particular variants that have been modified in the amino acid sequence without adversely affecting, to any substantial degree, the ability of the TTSE to form alpha helical coiled coil trimers. In various aspects of the invention, the trimeric polypeptide according to the invention includes a TTSE as a trimerizing domain having at least 66% amino acid sequence identity to the consensus sequence of SEQ ID NO: 47; for example at least 73%, at least 80%, at least 86% or at least 92% sequence identity to the consensus sequence of SEQ ID NO: 47 (counting only the defined (not X) residues). In other words, at least one, at least two, at least three, at least four, or at least five of the defined amino acids in SEQ ID NO: 47 may be substituted.

In one particular embodiment, the cysteine at position 50 (C50) of SEQ ID NO: 46 can be advantageously be mutagenized to serine, threonine, methionine or to any other amino acid residue in order to avoid formation of an unwanted inter-chain disulphide bridge, which can lead to unwanted multimerization. Other known variants include at least one amino acid residue selected from amino acid residue nos. 6, 21, 22, 24, 25, 27, 28, 31, 32, 35, 39, 41, and 42 (numbering according to SEQ ID NO: 46), which may be substituted by any non-helix breaking amino acid residue. These residues have been shown not to be directly involved in the intermolecular interactions that stabilize the trimeric complex between three TTSEs of native tetranectin monomers. In one aspect, the TTSE has a repeated heptad having the formula a-b-c-d-e-f-g (N to C), wherein residues a and d (i.e., positions 26, 33, 37, 40, 44, 47, and 51 may be any hydrophobic amino acid (numbering according to SEQ ID NO: 46).

In further embodiments, the TTSE trimerization domain may be modified by the incorporation of polyhistidine sequence and/or a protease cleavage site, e.g., Blood Coagulating Factor Xa or Granzyme B (see US 2005/0199251, which is incorporated herein by reference), and by including a C-terminal KG or KGS sequence. Also, to assist in purification, Proline at position 2 may be substituted with Glycine to assist in purification.

The terms “C-type lectin-like protein” and “C-type lectin” are used to refer to any protein present in, or encoded in the genomes of, any eukaryotic species, which protein contains one or more CTLDs or one or more domains belonging to a subgroup of CTLDs, the carbohydrate recognition domains (CRDs), which bind carbohydrate ligands. The definition specifically includes membrane attached C-type lectin-like proteins and C-type lectins, “soluble” C-type lectin-like proteins and C-type lectins lacking a functional transmembrane domain and variant C-type lectin-like proteins and C-type lectins in which one or more amino acid residues have been altered in vivo by glycosylation or any other post-synthetic modification, as well as any product that is obtained by chemical modification of C-type lectin-like proteins and C-type lectins.

The CTLD consists of roughly 120 amino acid residues and, characteristically, contains two or three intra-chain disulfide bridges. Although the similarity at the amino acid sequence level between CTLDs from different proteins is relatively low, the 3D-structures of a number of CTLDs have been found to be highly conserved, with the structural variability essentially confined to a so-called loop-region, often defined by up to five loops. Several CTLDs contain either one or two binding sites for calcium and most of the side chains which interact with calcium are located in the loop-region.

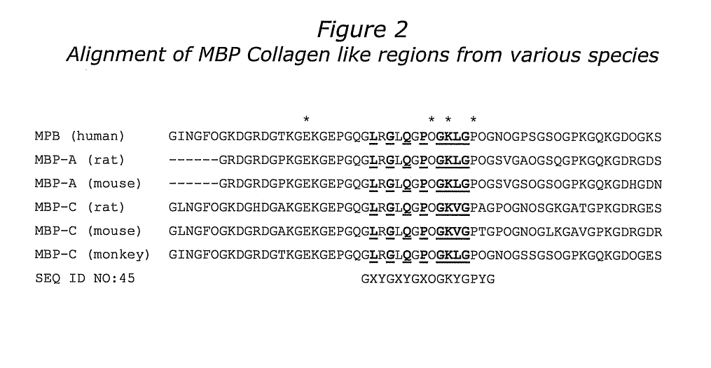

On the basis of CTLDs for which 3D structural information is available, it has been inferred that the canonical CTLD is structurally characterized by seven main secondary-structure elements (i.e. five β-strands and two α-helices) sequentially appearing in the order β1, α1, α2, β2, β3, β4, and β5. FIG. 2 illustrates an alignment of the CTLDs of known three dimensional structures of ten C-type lectins. In all CTLDs, for which 3D structures have been determined, the β-strands are arranged in two anti-parallel β-sheets, one composed of β1 and β5, the other composed of β2, β3 and β4. An additional β-strand, β0, often precedes β1 in the sequence and, where present, forms an additional strand integrating with the β1, ⊕5-sheet. Further, two disulfide bridges, one connecting α1 and β5 (C_{I}-C_{IV}) and one connecting β3 and the polypeptide segment connecting β4 and β5 (C_{II}-C_{III}) are invariantly found in all CTLDs characterized to date.

In the CTLD 3D-structure, these conserved secondary structure elements form a compact scaffold for a number of loops, which in the present context collectively are referred to as the “loop-region,” protruding out from the core. In the primary structure of the CTLDs, these loops are organized in two segments, loop segment A, LSA, and loop segment B, LSB. LSA represents the long polypeptide segment connecting β2 and β3 that often lacks regular secondary structure and contains up to four loops. LSB represents the polypeptide segment connecting the β-strands β3 and β4. Residues in LSA, together with single residues in β4, have been shown to specify the Ca²⁺- and ligand-binding sites of several CTLDs, including that of tetranectin. For example, mutagenesis studies, involving substitution of one or a few residues, have shown that changes in binding specificity, Ca²⁺-sensitivity and/or affinity can be accommodated by CTLD domains A number of CTLDs are known, including the following non-limiting examples: tetranectin, lithostatin, mouse macrophage galactose lectin, Kupffer cell receptor, chicken neurocan, perlucin, asialoglycoprotein receptor, cartilage proteoglycan core protein, IgE Fc receptor, pancreatitis-associated protein, mouse macrophage receptor, Natural Killer group, stem cell growth factor, factor IX/X binding protein, mannose binding protein, bovine conglutinin, bovine CL43, collectin liver 1, surfactant protein A, surfactant protein D, e-selectin, tunicate c-type lectin, CD94 NK receptor domain, LY49A NK receptor domain, chicken hepatic lectin, trout c-type lectin, HIV gp 120-binding c-type lectin, and dendritic cell immunoreceptor. See U.S. 2007/0275393, which is incorporated by reference herein in its entirety.

The expression “effective amount” refers to an amount of one or both of a fusion protein of the invention and a therapeutic agent which is effective for preventing, ameliorating or treating the disease or condition in question whether administered simultaneously or sequentially. In particular embodiments, an effective amount is the amount of the fusion protein, and a therapeutic agent in combination sufficient to enhance, or otherwise increase the propensity (such as synergistically) of a cell to undergo apoptosis, reduce tumor volume, or prolong survival of a mammal having a cancer or other disease.

The terms “cancer,” “cancerous,” and “malignant” refer to or describe the physiological condition in mammals that is typically characterized by unregulated cell growth. Examples of cancer include but are not limited to, carcinoma including adenocarcinoma, lymphoma, blastoma, melanoma, sarcoma, and leukemia. More particular examples of such cancers include squamous cell cancer, small-cell lung cancer, non-small cell lung cancer (NSCLC), gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer, colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer.

In the present context, the term “antibody” is used to describe an immunoglobulin whether natural or partly or wholly synthetically produced. As antibodies can be modified in a number of ways, the term “antibody” should be construed as covering any specific binding member or substance having a binding domain specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. The term also covers any polypeptide or protein having a binding domain which is, or is homologous to, an antibody binding domain, e.g. antibody mimics. These can be derived from natural sources, or they may be partly or wholly synthetically produced. Examples of antibodies are the immunoglobulin isotypes and their isotypic subclasses; fragments which comprise an antigen binding domain such as Fab, Fab′, F(ab′)₂, scFv, Fv, dAb, Fd; and diabodies.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gamma 11 and calicheamicin omega 11 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,22″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included in the definition are proteasome inhibitors such as bortezumib (Velcade), BCL-2 inhibitors, IAP antagonists (Smac synthetics), HDAC inhibitors (HDACI) and kinase inhibitors (Sorafenib).

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

Particular Aspects of the Invention

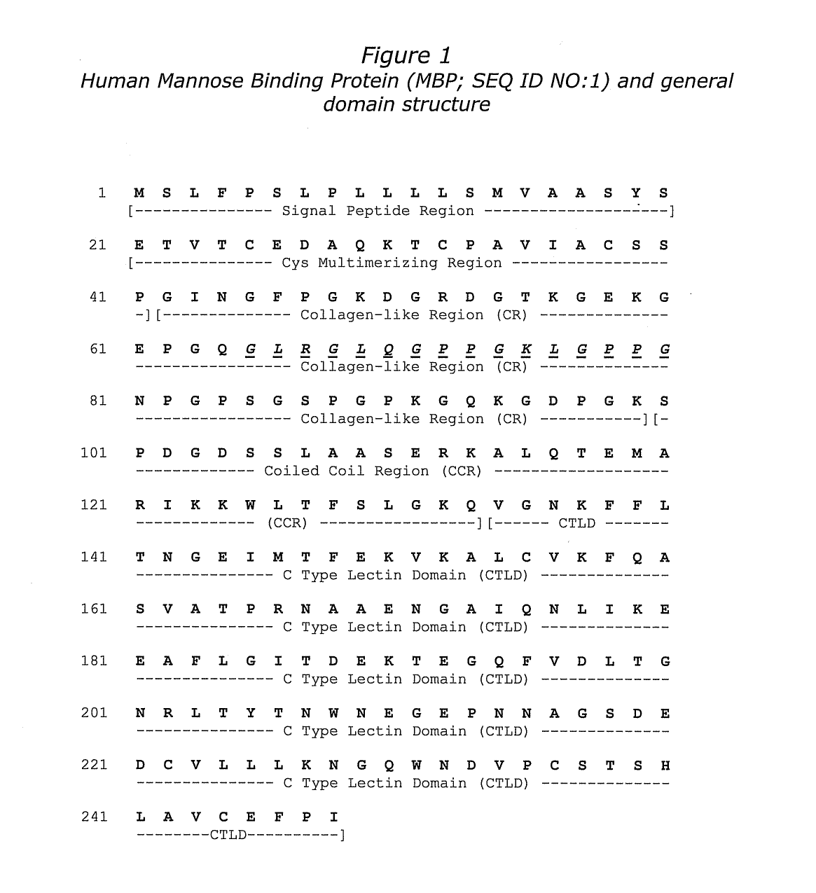

Turning now to the invention in more detail, full length human mannose binding protein (MBP) is disclosed in SEQ ID NO: 38 (FIG. 1). The various functional regions of MBP are also described in FIG. 1. As used herein, “mannose binding lectin (MBL) polypeptide” is taken to mean amino acids 21-133 of SEQ ID NO: 38 (also represented as SEQ ID NO: 48), as well as functional variants and fragments thereof, as described herein. The MBL polypeptide of the fusion protein is able to bind to mannose binding protein (MBP)-associated serine proteases (MASPs) and can initiate effector function, such as an immune response through complement fixation.

In certain embodiments the MBL polypeptide comprises SEQ ID NO: 48. In other embodiments the MBL polypeptide comprises amino acids 42-133 of SEQ ID NO: 38. In some embodiments the MBL polypeptide comprises amino acids 48-99 of SEQ ID NO: 38. In yet other embodiments the MBL polypeptide comprises amino acids 65-80 of SEQ ID NO: 38 (also represented as SEQ ID NO: 49). In a further embodiment the MBL polypeptide comprises variants of SEQ ID NO:3 having the general sequence of:

| | |
|---|---|
| GXYGXYGXOGKYGPYG | (SEQ ID NO: 45) |

wherein X and Y can be any amino acid, and 0 is hydroxyproline (HyP). In certain embodiments X is selected from Leu, Pro, Phe, Ser, H is, and Glu. In certain embodiments Y is selected from Arg, Ser, HyP, Gln, Leu, Val, Met, Ala, Thr, Lys, glycosylated Lys (g-Lys), and hydroxylated Lys (h-Lys).

In further embodiments, the MBL polypeptide variants can comprise amino acid substitutions in regions of SEQ ID NO: 48 other than described above in SEQ ID NO: 45. The MBL polypeptide variants of the invention retain the structure necessary for the binding sites for MASPs; thus, the variants of the invention do not disrupt the structure of the collagen-like domain of the MBL polypeptide. For example, see Wallis, et al., J. Biol. Chem., 279(14): 14065-14073 (2004), incorporated herein by reference. Accordingly, MBL variants can be derived from consensus sequences of various collagen-like regions, multimerizing regions, and coiled coil regions across multiple species. Further, conservative amino acid substitutions can be made based on secondary and tertiary structures of various MBL polypeptides, as hydropathy, charge, and hydrogen bonding interactions can all be taken into consideration, and appropriate substitutions made which retain MASP binding activity. In embodiments that comprise variants, such as deletion, insertion, or substitution variants in the region outside of the MASP binding region of the MBL polypeptide, the percent identity can be as low as 50%. In other embodiments comprising such variation within the MASP binding region, variants are at least 80% identical to SEQ ID NO: 38 or SEQ ID NO: 48. In certain embodiments such variants are at least 85%, 86%, 87%, 88%, 89%, 90%, 91%, 92%, 93%, 94%, 95%, 96%, 97%, 98%, or 99%, identical to SEQ ID NO: 38 or SEQ ID NO: 48. In various examples of the fusion protein of the invention, a targeting sequence is fused C-terminally to MBL at K123, K124, W125, and T127 of MBL (numbering relative to SEQ ID NO: 38).

In certain embodiments of the above aspect of the invention, the sequence of the second polypeptide can be selected to target moieties such as carbohydrates, lipids, and/or amino acid sequences associated with a particular cell type and/or pathogen. For example, the targeting sequence can be selected to target carbohydrates, lipids, and/or proteins (e.g., cell surface receptor or transmembrane transport proteins) associated cells such as, for example, dendritic cells, B cells, T cells, and/or tumor cells, or combinations thereof. Similarly, the targeting sequence can be selected to target carbohydrates, lipids, and/or proteins (e.g., cell surface receptor or transmembrane transport proteins) associated with pathogens such as, for example, virus, fungi, bacteria, protozoa, or other parasites, or combinations thereof.

In an embodiment the targeting sequence is selected to target carbohydrates, lipids, and/or proteins associated with tumor cells. In a particular embodiment the targeting sequence is targeted to at least one protein associated with a tumor cell such as the non-limiting examples of CA125, CA19-9, CA15-3, D97, gp100, Lewis Y, CD20, CD21, TAG-72, EGF receptor, Epithelial cell adhesion molecule (Ep-CAM), Carcinoembryonic antigen (CEA), Prostate specific antigen (PSA), PMSA, CDCP1, CD26, Hepsin, HGF (hepatocyte growth factor), Met, CAIX(G250), EphhB4 (Ephrin type-B receptor 4), EGFR1, EGFR2, PDGF, VEGFR, DPP6, syndecan 1, IGFBP2 (Human insulin-like growth factor binding protein 2), CD3, CD28, CTL4, VEGF, Her2/Neu receptor, tyrosinase, MAGE 1, MAGE 3, MART, BAGE, TRP-1, CA 50, CA 72-4, MUC 1, NSE (neuron specific enolase), α-fetoprotein (AFP), SSC (squamous cell carcinoma antigen), BRCA-1, BRCA-2, glypican-3 (GPC3), colon antigen-1 (COA-1), six transmembrane epithelial antigen of the prostate 1 (STEAP1), NY-ESO-1, podoplanin, melanoma-overexpressed antigen (meloe), CD200 and hCG.

In an embodiment the targeting sequence is selected to target carbohydrates, lipids, and/or proteins associated with dendritic cells. “Dendritic cells” as used herein includes any known dendritic cell subtype such as, for example, myeloid or plasmacytoid dendritic cells. In a particular embodiment the targeting sequence is targeted to at least one protein associated with dendritic cells such as the non-limiting examples of CD83, CD205, CD197, CCR7 and CD209/DC-SIGN.

In an embodiment the targeting sequence is selected to target carbohydrates, lipids, and/or proteins associated with B cells. In a particular embodiment the targeting sequence is targeted to at least one protein associated with dendritic cells such as the non-limiting examples of CD19, CD20, CD21, CD22, CD32, CD79α, CD79β, CD83, CD138, CD139, CD179α, CD179β, and CD180, TACI, BCMA, and BR-3.

In an embodiment the targeting sequence is selected to target carbohydrates, lipids, and/or proteins associated with T cells. “T cells” as used herein includes any type of T cell subtype such as, for example, activated T cells; regulatory T cells (T_{REG}); cytotoxic T cells (T_{C}, CTL); helper T cells (effector T cells or T_{H}; e.g., T_{H}1, T_{H}2, T_{H}3, T_{H}17, T_{H}F); memory T cells (T_{CM}, T_{EM}); natural killer T cells (NKT); gamma delta T cells (γδ T cells). In a particular embodiment the targeting sequence is targeted to at least one protein associated with T cells such as the non-limiting examples of CD3, CD4, CD8, CCR7, CD153, CD154, CD137, CD134, CD25, CD28, CD129, CD200, CDw217, α-TCR, and (3-TCR.

In an embodiment the targeting sequence is selected to target carbohydrates, lipids, and/or proteins associated with at least one pathogen (bacteria, protozoa, parasites, viruses, and the like). In a particular embodiment the targeting sequence is targeted to at least one protein associated with a pathogen such as the non-limiting examples of respiratory syncytial viral (RSV) proteins (e.g., RSV F glycoprotein, RSV G glycoprotein, and the like); human parainfluenza viruses 1-4; human metapneumovirus, hendravirus and nipahvirus, and the orthomyxoviridae, such as influenza group A, B or C viral proteins, e.g., influenza virus neuramimidase and influenza virus hemagglutinin; herpesviridae, such as herpes simplex viral proteins, e.g., herpes simplex virus 1 or 2 glycoproteins including for example, gB, gC, gD, or gE proteins, or homologues of gB, gC, gD, and gE or other proteins from cytomegalovirus, Epstein-Barr virus, varicella zoster virus, and human herpesviruses 6, 7, or 8, or from monkey B-virus (cercopithecine herpesvirus 1). Proteins also include but are not limited to proteins from the retroviridae, such as human immunodeficiency virus types 1 and 2 (HIV1 and HIV2) and human T-lymphotrophic viruses 1-4 (HTLV-1-4) envelope and other proteins, and proteins from betaretroviruses and spumaviruses. Proteins may also be from the togaviridae, including proteins such as the capsid proteins from rubella or the alphaviruses, e.g., Venezuelan equine encephalitis virus, Eastern equine encephalitis viruses, and Western equine encephalitis viruses and the Semliki forest complex of viruses. Proteins also include but are not limited to proteins from the adenoviridae (e.g., human adenoviruses A-F, and other miscellaneous human adenoviruses), from the poxyiridae, e.g. variola (smallpox), vaccinia, cowpox, monkeypox, Molluscum contagiosum, tanapox, yaba monkey tumor virus, orf virus, pseudocowpox, and bovine papular stomatitis virus. Proteins also include but are not limited to proteins from the parvoviridae, such as B19 virus, adeno-associated virus, and human bocaviruses. Proteins also include but are not limited to proteins from the papillomaviridae, including proteins from the numerous different human papillomaviruses. Proteins also include but are not limited to proteins from the polyomaviridae, such as JC virus, BK virus, KI virus, WU virus and Merkel cell polyoma viruses, and from circoviridae, such as transfusion transmitted virus (TTV). Such proteins also include but are not limited to proteins from the reoviridae, such as rotaviruses, orthoreoviruses, and coltivirus; hepadnaviridae (e.g., hepatitis B virus); the picornaviridae, such as enteroviruses, echoviruses, parechoviruses, coxsackie A and B group viruses, poliovirus, hepatitis A virus, cardioviruses, and rhinoviruses, the flaviviridae, such as hepatitis C virus and Dengue fever virus, yellow fever virus, Japanese encephalitis virus, Kyasanur Forest disease virus, Murray Valley encephalitis virus, St. Louis encephalitis virus, and tick-borne encephalitis virus, the coronaviridae, such as human coronaviruses, human toroviruses, and SARS coronavirus proteins, and including but not limited to the S and M proteins from coronaviridae, the bunyaviridae, such as Crimean-Congo hemorrhagic fever virus, California encephalitis virus, La Crosse virus, Rift Valley fever virus, and numerous human-transmissible hantaviruses, the bornaviridae, such as Borna disease virus, the rhabdoviridae, such as rabies virus, and the filoviridae, such as ebolaviruses and Marburg virus, the caliciviridae, such as noroviruses (e.g. Norwalk virus), sapoviruses (e.g. Sapporo viruses), and other caliciviruses, the astroviridae, such as human astroviruses, the arenaviridae, such as lymphocytic choriomeningitis virus, Lassa virus, Junin virus, Machupo virus, Guanarito virus, Savia virus, Tacaribe virus, Flexal virus, and Whitewater Arroyo virus, the hepeviridae, such as hepatitis E virus, and surface proteins derived from the genomes of deltaviruses, such as hepatitis delta virus.

Surface proteins of bacteria also include, but are not limited to, those found on various species of alphaproteobacteria, such as those of the genera Anaplasma (including Anaplasma phagocytophilum), Ehrlichia (including E. chaffeensis and E. erwingii), Rickettsia (including R. prowazekii, R. typhi, and R. rickettsii), Bartonella (including B. henselae), and Brucella, the betaproteobacteria, such as those of the genera Burkholderia (including B. cepacia and B. pseudomallei), Bordetella (including B. pertussis), and Neisseria (including N. gonorrhoeae and N. meningitides), the gammaproteobacteria, such as those of the genera Francisella (including F. tularensis), Legionella (including L. pneumophila), Coxiella (including C. burnetii), Acinetobacter (including A. baumannii), Moraxella (including M. lacunata), Pseudomonas (including P. aeruginosa and P. oryzihabitans), Providencia (including P. stuartii), Vibrio (including V. cholerae, V. vulnificus, and V. parahaemolyticus), Citrobacter, Enterobacter (including E. cloacae and E. aerogenes), Escherichia (including E. coli O157:H7), Klebsiella (including K. pneumoniae), Proteus (including P. vulgaris, P. mirabilis, and P. penneri), Salmonella (including S. enterica serovars Typhimurium, Enterititidis, and Typhi), Serratia (including S. marcescens) Shigella (including S. flexneri, S. dysenteriae, and S. sonnei), Yersinia (including Y. pestis), Haemophilus (including H. influenzae and H. ducreyi), and Pasteurella (including P. multocida), the epsilonproteobacteria, such as those of the genera Campylobacter (including C. jejuni, C. coli, and C. fetus) and Helicobacter (including H. pylori), the firmicutes, such as those of the genera Clostridium (including C. difficile, C. perfringens, C. botulinum, C. sordellii, and C. tetani), Mycoplasma (including M. pneumoniae), Bacillus (including B. anthracis and B. cereus), Listeria (including L. monocytogenes), Staphylococcus (including S. aureus, S. saprophyticus, and S. epidermis), Enterococcus (including E. faecalis and E. faecium), and Streptococcus (including S. pyogenes, S. pneumoniae, S. agalactiae, S. mutans, S. viridans, and S. dysgalactiae), the actinobacteria, such as those of the genera Actinomyces (including A. israelii), Corynebacterium (including C. diphtheriae, C. amycolatum, and C. parvum), Gardnerella (including G. vaginalis), Mycobacterium (including M. tuberculosis, M. leprae, M. abscessus, and the M. avium complex), and Nocardia (including N. asteroides), the chlamydiae, such as those of the genera Chlamydia (including C. trachomatis) and Chlamydophila (including C. psittaci and C. pneumoniae), the spirochaetes, such as those of the genera Borrelia (including B. burgdorferi), Leptospira (including L. interrogans), and Treponema (including T. pallidum), the bacterioidetes, such as those of the genera Bacteroides and Prevotella, and the fusobacteria, such as those of the genus Fusobacterium.

Surface proteins of parasites include, but are not limited to, those found on various life stages (spore, hyphae, bud, yeast) of species of fungi, such as Aspergillus fumigatus, A. flavus, A. terreus, A. nidulans, A. niger, Blastomyces dermatidis, Candida spp., Coccidioides spp., Cryptococcus neoformans, C. gatti, Brachiola algerae, B. connori, B. vesicularum, Encephalitozoon cuniculi, E. hellem, E. intestinalis, Enterocytozoon bieneusi, Microsporidium ceylonensis, M. africanum, Nosema ocularum, Pleistophora spp., Trachipleistophora hominis, T. anthropophthera, Vittaforma corneae, and Pneumocystis jirovecii (formerly classified as P. carinii) and on various life stages of species of apicomplexan protozoa, such as Babesia microti, B. divergens, Cryptosporidium parvum, C. hominis, C. felis, C. canis, C. muris, C. meleagridis, Cyclospora cayetanensis, Isospora belli, Plasmodium falciparum, P. malariae, P. ovale, P. vivax, and Toxoplasma gondii, as well as on species of ciliophoran protozoa such as Balantidium coli, and euglenozoans such as Leishmania chagasi, L. donovani, L. infantum, L. mexicana, L. amazonensis, L. venezuelensis, L. tropica, L. major, L. aethiopica, L. (subgenus Viannia) braziliensis, L. (V.) guyanensis, L. (V.) panamensis, and L. (V.) peruviana, Trypanosoma cruzi, T. brucei rhodesiense, and T. brucei gambiense, and on species of amoebozoans such as Acanthamoeba spp., Balamuthia mandrillaris, and Entamoeba histolytica, on species of diplomonads such as Giardia lamblia, on species of trichomonads such as Dientamoeba fragilis and Trichomonas vaginalis, on protists such as Naegleria fowleri, on species of stramenopiles such as Blastocystis hominis, on various life stages of species of nematodes such as Angiostrongylus cantonensis, A. costaricensis, Anisakis simplex, Pseudoterranova decipiens, Ascaris lumbricoides and Ascaris spp., Baylisascaris proconyis, Capillaria phillipinensis, C. hepatica, C. aerophile, Dracunculus medicinensis, Brugia malayi, B. timori, Dirofilaria spp., Loa loa, Mansonella ozzardi, M. perstans, M. streptocerca, Wucheria bancrofti, Enterobius vermicularis, E. gregorii, Gnathostoma spinigerum, G. hipidum, Ancylostoma duodenale, A. ceylanicum, A braziliense, A. caninum, Uncinaria stenocephala, Necator americanus, Onchocerca volvulus, Strongyloides stercoralis, S. fuelleborni, Toxocara canis, T. cati, Trichinella spiralis, T pseudospiralis, T. nativa, T. nelsoni, T. britovi, and Trichuris trichiura, and on various life stages of species of platyhelminthes such as Clonorchis sinensis, Diphyllobothrium latum, D. pacificum, D. crodatum, D. ursi, D. dendriticum, D. lanceolatum, D. dalliae, D. yonagoensis, Dipylidium caninum, Echinococcus multilocularis, Fasciola hepatica, F. gigantus, Faciolopsis buski, Heterophyes heterophyes, Hymenolepis nana, Metagonimus yokogawai, Opisthorchis viverrini, O. felineus, Paragonimus westermani, Paragonimus spp., Schistosoma mansoni, S. haematobium, S. japonicum, S. mekongi, S. intercalatum, and Taenia solium and Taenia spp.

The targeting sequence can comprise any sequence that has binding affinity to any of the above-mentioned non-limiting examples of target moieties. Some non-limiting examples of targeting sequences include lectin domains (e.g., C-type lectin domains (CTLDs)), antibody sequences and antigen binding fragments thereof (e.g., scFv, Fab′, Fab₂′, etc.), or other alternative scaffold structures the exhibit binding affinity for a particular carbohydrate, lipid, and or protein.

In certain embodiments the fusion protein of the invention takes advantage of the binding characteristics of C-type lectin domains (CTLDs) and the complement fixation characteristics of MBL. Calcium-dependent lectins (C-type lectins) are expressed in a large number of cell types including macrophages, B- and T-lymphocytes, mast cells, and natural killer (NK) cells. Macrophage lectin proteins perform a variety of functions in the recognition and destruction of foreign cells and pathogens. Gram positive and Gram negative bacteria have been shown to interact with C-type lectins [Athamna et al., Infect Immun. 59:1673 (1991); Shimaoka et al., J. Immunol. 166(8):5108 (2001)]. A human macrophage C-type lectin has been found to recognize Tn Ag, a well-known human carcinoma-associated epitope [Suzki et al., J Immunol 156:128 (1996)]. Furthermore, the recombinant cytosolic carbohydrate binding domain of the mouse macrophage C-type lectin also served as an inhibitor of cytotoxic activity, indicating that the lectin was a direct mediator of the macrophage tumoricidal response [Imai et al., J Immunol Methods 171:23 (1994)]. Unique macrophage lectins may specifically interact with surface antigens expressed by certain abnormal or diseased cells. The lectins may direct the macrophages to abnormal or diseased cells. C-type lectins are glycoproteins that exhibit amino acid sequence similarities in their carbohydrate recognition domains (CRD) and that bind to selected carbohydrates in a calcium-dependent manner. C-type lectins can be classified in four general categories [Vasta et al., Ann N Y Acad. Sci., 712:55-73 (1994); Spiess, Biochemistry, 29:10009-10018 (1990)]. The first category comprises type II membrane-integrated proteins, such as asialoglycoprotein receptors, macrophage galactose and N-acetyl glucosamine (GlcNac)-specific lectin, and CD23 (Fc-εRII). Many members in this group exhibit specificity for galactose/fucose, galactosamine/GalNac or GlcNac residues. The second category includes cartilage and fibroblast proteoglycan core proteins. The third category includes the collectins, which include MBP, pulmonary surfactant protein SP-A, and conglutinin. The fourth group includes certain adhesion molecules, which are known as LEC-CAMs (e.g., MeI-14, GMP-140, and ELAM-1).

C-type lectins are known to function as agglutinins, opsonins, complement activators, and cell-associated recognition molecules [Vasta et al., Ann N Y Acad. Sci., 712:55-73, 1994; Spiess, Biochemistry, 29:10009-10018, 1990; Kery, Int J Biochem., 23(7-8):631-40, 1991]. For instance, macrophage mannose receptors serve a scavenger function (Shepherd et al., Am J Respir Cell Mol. Biol., 2(4):335-40, 1990), as well as mediating the uptake of pathogenic organisms, including Pneumocystis carinii [Ezekowitz et al., Nature, 351(6322):155-8, 1991) and Candida albicans (Ezekowitz et al. J Exp Med., 172(6):1785-94, (1990)]. Thus, C-type lectins exhibit diverse functions with biological significance, and possess desirable binding characteristics to particular target moieties, including particular cell types and pathogens.

Any type of functional CTLD can be used as the second polypeptide in the fusion protein of the invention. In one embodiment, the targeting sequence comprises a sequence that targets a moiety on the surface of a tumor cell such as, for example, a Lewis antigen. In yet a further embodiment the targeting sequence comprises DC-Sign (Dendritic Cell specific ICAM-3 grabbing nonintegrin), or functional fragment or variant thereof that binds to a Lewis antigen.

In one aspect the invention is directed to a fusion protein of an MBL, the targeting sequence and a tetranectin trimerizing domain. In accordance with the invention, the targeting sequence may either be linked to the N- or the C-terminal amino acid residue of tetranectin trimerizing domain. In various embodiments, the targeting sequence is attached to the N-terminal or the C-terminal, and the MBL polypeptide having effector function is bound to the other terminus.

In addition to be terminally linked via a peptide bond, the heterologous targeting sequence be attached to the MBL complex according to other techniques. For example, via a peptide bond to a side chain or via a bond to a cysteine residue. But any way of coupling covalently heterologous material to a polypeptide chain will be useful. The skilled person will know of such possibilities, e.g. by consulting the teachings of WO 95/31540 in this regard which are hereby incorporated by reference.

In another aspect, the invention provides a method of activating a mammalian complement system comprising administering to the mammal a fusion protein comprising a first polypeptide comprising a mannose binding lectin (MBL) polypeptide, wherein the first polypeptide has effector function; and a second polypeptide comprising a sequence that binds to a targeted moiety.

In an aspect, the invention provides a pharmaceutical composition comprising a fusion protein comprising a first polypeptide comprising a mannose binding lectin (MBL) polypeptide, wherein the first polypeptide has effector function; and a second polypeptide comprising a sequence that binds to a targeted moiety, and a pharmaceutically acceptable excipient.

In an aspect the invention provides a method of treating a pathogenic disease comprising administering to a patient suffering from the disease a fusion protein, or a pharmaceutical composition thereof, comprising a first polypeptide comprising a mannose binding lectin (MBL) polypeptide, wherein the first polypeptide has effector function; and a second polypeptide comprising a sequence that binds to a targeted moiety, wherein the targeted moiety is a cell surface receptor of the pathogen.

In an aspect the invention provides a method of treating a proliferative disease comprising tumor cells comprising administering to a patient in need thereof a fusion protein, or a pharmaceutical composition thereof, comprising a first polypeptide comprising a mannose binding lectin (MBL) polypeptide, wherein the first polypeptide has effector function; and a second polypeptide comprising a sequence that binds to a targeted moiety, wherein the targeted moiety is a receptor on the surface of a tumor cell. In an embodiment the receptor on the surface of a tumor cell comprises a Lewis antigen. In a further embodiment the second polypeptide comprises DC-Sign (Dendritic Cell specific ICAM-3 grabbing nonintegrin), or functional fragment or variant thereof that binds to a Lewis antigen.

As a proof of concept, a non-limiting embodiment of the invention is described in detail in the Examples, wherein the targeting sequence comprises DC-Sign, which is a type II transmembrane protein belonging to the C-type lectin family. The protein is expressed on the surface of dendritic cells (DC) in the periphery and participates in the primary contact between the antigen-presenting cells and resting T-cells in the lymphatic system via ICAM-3 on the T-cells. DC-Sign also interacts with ICAM-2 on epithelial cells during migration of DCs to lymphoid tissues. DC-Sign also binds strongly to the HIV envelope protein gp120 and facilitates viral infection in trans of CD4+ T-cells. The DC-Sign protein consists of a short amino-terminal cytoplasmic tail, a transmembrane domain, a stalk of up to 7½ repeats, followed by a C-terminal C-type carbohydrate recognition domain (CRD). The stalk promotes the formation of tetramers (coiled coil).

Blood group-related Lewis tumor antigens (e.g. Le^{x} and Le^{y}) are expressed on the majority of human cancers of epithelial origin. Lewis antigens are complex oligosaccharides and are both found as glycolipids, embedded in the cell membrane and linked to cell surface proteins (e.g. HER1, HER2 and CEA) with only limited expression on normal tissue. Lewis antigens have been shown to mediate dendritic cell adhesion and tumor cell infiltration. Lewis Y interacts with the DC-SIGN receptor on dendritic cells to escape immune surveillance by promoting immune suppression. Thus, Lewis antigens provide a non-limiting example of a targeted moiety for the second polypeptide that comprises the fusion protein of the invention. The peptide and corresponding C-DNA sequence encoding the peptide of human MBL is provided in FIG. 1 and in the sequence listing, respectively.

In certain embodiments, the present invention provides combinations of an MBL polypeptide and DC-Sign as a fusion protein, which uniquely combine the ability of DC-SIGN to bind to certain Lewis antigens and the ability of MBP to activate the complement system. Further, careful comparisons of the MBP neck region and the DC-SIGN tetramerization domain (both helical coil-coil structures) identified regions with similar structural architecture. By preserving the helix rhythm, a number of different non-limiting MBP/DC-SIGN fusion proteins have been designed and are described in the Examples below.

The MBP/DC-SIGN fusion proteins of the present invention have a high order of multimerization which adds dramatically to the avidity gain (i.e., increased binding strength to cells expressing a high number of receptors) which increases the effect of the therapeutic molecule specifically targeting cancer cells and reduces the risk of side effects. In one embodiment, the fusion proteins may also block the interaction between the cancer cells and dendritic cells (this interaction leads to escape of immunosurveillance), and block Lewis antigen-mediated adhesion and tumor cell invasion. In a further embodiment, the fusion proteins of the invention can have the advantage of mediating killing of the targeted cells by complement activation and/or uptake by monocytes and neutrophils.

The invention also provides MBP/DC-SIGN fusion proteins, including the fusion proteins selected from MBP/DC-Sign CTLD-ABs (SEQ ID NO: 2), MBP/DC-Sign CTLD-ACs (SEQ ID NO: 4), MBP/DC-Sign CTLD-ADs (SEQ ID NO: 6), MBP/DC-Sign CTLD-ABsC (SEQ ID NO: 8), MBP/DC-Sign CTLD-ACsC (SEQ ID NO:10), MBP/DC-Sign CTLD-ADsC (SEQ ID NO:12), MBP/DC-Sign CTLD-FE (SEQ ID NO:14), MBP/DC-Sign CTLD-GE (SEQ ID NO:16), and MBP/DC-Sign CTLD-HE SEQ ID NO:18), MBP/DC-Sign CTLD-ACsCSG (SEQ ID NO:20), MBP/DC-Sign CTLD-ACsCSGGS (SEQ ID NO:22), and MBP/DC-Sign CTLD-ACsCSGGGS (SEQ ID NO:24), MBP/DC-Sign CTLD-ABs0 (SEQ ID NO:26) and MBP/DC-Sign CTLD-ABsC0 (SEQ ID NO:28).

In certain embodiments the fusion protein of the invention may additionally be linked to a third polypeptide, i.e. a third fusion partner. It may be that by adding such a third fusion partner to the MBP/DC-SIGN fusion protein of the invention, high yields of the MBP/DC-SIGN fusion protein can be obtained. The third fusion partner can, in accordance with the invention, be of any suitable kind provided that it is a peptide, oligopeptide, polypeptide or protein, including a di-peptide, a tri-peptide, a tetra-peptide, penta-peptide and a hexa-peptide. The third fusion partner can in certain embodiments be a single amino acid. It can also be selected such that it renders the fusion protein more resistant to proteolytic degradation, facilitates enhanced expression and secretion of the fusion protein, improves solubility, and/or allows for subsequent affinity purification of the fusion protein.

In certain embodiments the junction region between the fusion protein of the invention and a third fusion partner such as ubiquitin, comprises a Granzyme B protease cleavage site such as human Granzyme B (E.C. 3.4.21.79). More detailed information on the use of Granzyme B as fusion protein cleaving agent may be found in Published US Patent Application No. 2006/0199251 or WO/2004/094478, each incorporated herein by reference.

The third fusion partner may in further embodiments be coupled to an affinity-tag, or can also itself be an affinity tag. Such an affinity tag can include an affinity domain which permits the purification of the fusion protein on an affinity resin. The affinity-tag may be a polyhistidine-tag including hexahis-tag, a polyarginine-tag, a FLAG-tag, a Strep-tag, a c-myc-tag, a S-tag, a calmodulin-binding peptide, a cellulose-binding peptide, a chitin-binding domain, a glutathione 5-transferase-tag, or a maltose binding protein, or any other affinity tag known to those of skill in the art.

In yet further embodiments, the third fusion partner can comprise a molecule that stabilizes the fusion protein by increasing (extending) the half-life of the fusion protein. Molecules that can extend the half-life of a biomolecule, such as a protein, are known by those of skill in the art and include, for example, a BSA-binding peptide, various polyols (e.g. PEGs), IgG-binding peptides or peptides binding to FcRn or an Fc antibody fragment.

The method of the invention can in certain embodiments include an isolation step for isolating the MBP/DC-SIGN fusion protein of the invention which is formed by the enzymatic cleavage of the fusion protein, which has e.g. been immobilized by the use of the above mentioned affinity-tag systems. This isolation step can be performed by any suitable means known in the art for protein isolation, including the use of ion exchange and fractionation by size, the choice of which depending on the character of the fusion protein. In an embodiment, the region between the third fusion partner and the region comprising the MBL polypeptide and the DC-SIGN polypeptide is contacted with the human serine protease Granzyme B to cleave of the fusion protein at a Granzyme B protease cleavage site to yield the fusion protein of the invention.

The present invention further provides an isolated nucleic acid encoding a MBP/DC-SIGN fusion protein of the present invention. Nucleic acids include DNA and RNA. More specifically, there is provided isolated nucleic acid which encodes MBP/DC-SIGN fusion proteins according to the invention including a nucleotide sequence selected from the group consisting of SEQ ID NO: 1, SEQ ID NO: 3, SEQ ID NO: 5, SEQ ID NO: 7, SEQ ID NO: 9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 and SEQ ID NO:27.

The invention also provides nucleic acid constructs in the form of plasmids, vectors, transcription or expression cassettes which comprise at least one nucleic acid as described above. Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press.

The present invention also provides a recombinant host cell which comprises on or more constructs as above. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. In one embodiment the host cell is HEK293 cells.

The therapeutic application of the polypeptides of the present invention comprises use of the MBP/DC-SIGN fusion protein for the treatment of cancer diseases, including breast cancer, prostate cancer, ovarian cancer, gastric cancer, lung cancer, liver cancer, myeloid cancer and epithelial cancer.

HERCEPTIN® (trastuzumab), which binds selectively to HER2 (Erb B2), has been approved for the treatment of breast cancer in tumors that overexpress HER2, which glycosylated with Lewis antigens on the cancer cells. Targeting of the Lewis antigen on HER2 will not interfere with the binding of HERCEPTIN®, and it is expected that the combination of the fusion protein of the present invention and HERCEPTIN® will have a synergetic effect on the treatment. Other therapeutic agents include monoclonal antibodies such as Rituximab, VEGF or EGFR-targeting agents, kinase inhibitors, immune stimulators, and cancer vaccines.

Chemotherapeutic drugs are commonly used for the treatment of colorectal cancer, such as 5-fluorouracil, mitomycin-C, oxaliplatin and Raltitrexed. Such compounds have been reported to enhance Lewis Y expression, which indicates that targeting of the Lewis antigen with the fusion protein of the present invention could work in synergy with an array of chemotherapeutic drugs.

Accordingly, the administration of the MBP/DC-SIGN fusion protein may comprise the administration of at least one further therapeutic agent, such as HERCEPTIN®, and chemotherapeutic agents such as Raltitrexed, Doxorubicin, taxol, 5-Fluorouracil, Irinotecan and Cisplatin, Mitomycin-C and oxaliplatin.

Methods of Treatment

Another aspect the invention relates to a method of treating a disease associated with an immune cell, a pathogenic cell, a tumor cell or a cell infected with a virus. The method includes contacting the cell with the fusion protein of the invention.

Another aspect of the invention is directed to a combination therapy. Formulations comprising the fusion protein and therapeutic agents are also provided by the present invention. It is believed that such formulations will be particularly suitable for storage as well as for therapeutic administration. The formulations may be prepared by known techniques. For instance, the formulations may be prepared by buffer exchange on a gel filtration column.

The fusion proteins and therapeutic agents can be administered in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Optionally, administration may be performed through mini-pump infusion using various commercially available devices.

Effective dosages and schedules for administering the fusion proteins may be determined empirically, and making such determinations is within the skill in the art. Single or multiple dosages may be employed. It is presently believed that an effective dosage or amount of the fusion proteins used alone may range from about 1 μg/kg to about 100 mg/kg of body weight or more per day. Interspecies scaling of dosages can be performed in a manner known in the art, e.g., as disclosed in Mordenti et al., Pharmaceut. Res., 8:1351 (1991).

When in vivo administration of the fusion proteins is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature [see, for example, U.S. Pat. No. 4,657,760; 5,206,344; or 5,225,212]. One of skill will appreciate that different formulations will be effective for different treatment compounds and different disorders, that administration targeting one organ or tissue, for example, may necessitate delivery in a manner different from that to another organ or tissue. Those skilled in the art will understand that the dosage of the fusion protein that must be administered will vary depending on, for example, the mammal which will receive the fusion protein agonist, the route of administration, and other drugs or therapies being administered to the mammal.

It is contemplated that yet additional therapies may be employed in the methods. The one or more other therapies may include but are not limited to, administration of radiation therapy, cytokine(s), growth inhibitory agent(s), chemotherapeutic agent(s), cytotoxic agent(s), tyrosine kinase inhibitors, ras farnesyl transferase inhibitors, angiogenesis inhibitors, and cyclin-dependent kinase inhibitors or any other agent that enhances susceptibility of cancer cells to treatment with the fusion proteins.

Preparation and dosing schedules for chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent may precede, or follow administration of the Apo2L variant, or may be given simultaneously therewith.

The fusion proteins and therapeutic agents (and one or more other therapies) may be administered concurrently (simultaneously) or sequentially. In particular embodiments, a fusion protein and a therapeutic agent are administered concurrently. In another embodiment, a fusion protein or trimeric complex is administered prior to administration of a therapeutic agent. In another embodiment, a therapeutic agent is administered prior to a fusion protein or trimeric complex. Following administration, treated cells in vitro can be analyzed. Where there has been in vivo treatment, a treated mammal can be monitored in various ways well known to the skilled practitioner. For instance, tumor tissues can be examined pathologically to assay for cell death or serum can be analyzed for immune system responses.

Pharmaceutical Compositions

The fusion protein according to the invention may be used for the preparation of a pharmaceutical composition by any suitable method well known in the art. The composition may together with the fusion protein, comprise one or more acceptable carriers therefore, and optionally other therapeutic and/or chemotherapeutic agents ingredients. Accordingly, the invention relates to a pharmaceutical composition comprising a therapeutically effective amount of the fusion protein of the invention along with a pharmaceutically acceptable carrier or excipient. As used herein, “pharmaceutically acceptable carrier” or “pharmaceutically acceptable excipient” includes any and all solvents, dispersion media, coating, antibacterial and antifungal agents, isotonic and absorption delaying agents, and the like that are physiologically compatible. Examples of pharmaceutically acceptable carriers or excipients include one or more of water, saline, phosphate buffered saline, dextrose, glycerol, ethanol and the like as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the of the antibody or antibody portion also may be included. Optionally, disintegrating agents can be included, such as cross-linked polyvinyl pyrrolidone, agar, alginic acid or a salt thereof, such as sodium alginate and the like. In addition to the excipients, the pharmaceutical composition can include one or more of the following, carrier proteins such as serum albumin, buffers, binding agents, sweeteners and other flavoring agents; coloring agents and polyethylene glycol.

The compositions can be in a variety of forms including, for example, liquid, semi-solid and solid dosage forms, such as liquid solutions (e.g. injectable and infusible solutions), dispersions or suspensions, tablets, pills, powders, liposomes and suppositories. The preferred form will depend on the intended route of administration and therapeutic application. In an embodiment the compositions are in the form of injectable or infusible solutions, such as compositions similar to those used for passive immunization of humans with antibodies. In an embodiment the mode of administration is parenteral (e.g., intravenous, subcutaneous, intraperitoneal, intramuscular). In an embodiment, the fusion protein (or trimeric complex) is administered by intravenous infusion or injection. In another embodiment, the fusion protein or trimeric complex is administered by intramuscular or subcutaneous injection.

Other suitable routes of administration for the pharmaceutical composition include, but are not limited to, rectal, transdermal, vaginal, transmucosal or intestinal administration.

Therapeutic compositions are typically sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e. fusion protein or trimeric complex) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

An article of manufacture such as a kit containing fusion proteins and therapeutic agents useful in the treatment of the disorders described herein comprises at least a container and a label. Suitable containers include, for example, bottles, vials, syringes, and test tubes. The containers may be formed from a variety of materials such as glass or plastic. The label on or associated with the container indicates that the formulation is used for treating the condition of choice. The article of manufacture may further comprise a container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use. The article of manufacture may also comprise a container with another active agent as described above.

Typically, an appropriate amount of a pharmaceutically-acceptable salt is used in the formulation to render the formulation isotonic. Examples of pharmaceutically-acceptable carriers include saline, Ringer's solution and dextrose solution. The pH of the formulation is preferably from about 6 to about 9, and more preferably from about 7 to about 7.5. It will be apparent to those persons skilled in the art that certain carriers may be more preferable depending upon, for instance, the route of administration and concentrations of fusion protein and Therapeutic agent.

Therapeutic compositions can be prepared by mixing the desired molecules having the appropriate degree of purity with optional pharmaceutically acceptable carriers, excipients, or stabilizers [Remington's Pharmaceutical Sciences, 16th edition, Osol, A. ed. (1980)], in the form of lyophilized formulations, aqueous solutions or aqueous suspensions. Acceptable carriers, excipients, or stabilizers are preferably nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, colloidal silica, magnesium trisilicate, polyvinyl pyrrolidone, and cellulose-based substances. Carriers for topical or gel-based forms include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations.

Formulations to be used for in vivo administration should be sterile. This is readily accomplished by filtration through sterile filtration membranes, prior to or following lyophilization and reconstitution. The formulation may be stored in lyophilized form or in solution if administered systemically. If in lyophilized form, it is typically formulated in combination with other ingredients for reconstitution with an appropriate diluent at the time for use. An example of a liquid formulation is a sterile, clear, colorless unpreserved solution filled in a single-dose vial for subcutaneous injection.

Therapeutic formulations generally are placed into a container having a sterile access port, for example, an intravenous solution bag or vial having a stopper pierceable by a hypodermic injection needle. The formulations are preferably administered as repeated intravenous (i.v.), subcutaneous (s.c.), intramuscular (i.m.) injections or infusions, or as aerosol formulations suitable for intranasal or intrapulmonary delivery (for intrapulmonary delivery see, e.g., EP 257,956).

The molecules disclosed herein can also be administered in the form of sustained-release preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res., 15: 167-277 (1981) and Langer, Chem. Tech., 12: 98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the Lupron Depot (injectable microspheres composed of lactic acid-glycolic acid copolymer and leuprolide acetate), and poly-D-(−)-3-hydroxybutyric acid (EP 133,988).

Production of Fusion Proteins

The fusion protein of the invention can be expressed in any suitable standard protein expression system by culturing a host transformed with a vector encoding the fusion protein under such conditions that the fusion protein is expressed. Preferably, the expression system is a system from which the desired protein may readily be isolated and refolded in vitro. As a general matter, prokaryotic expression systems are preferred since high yields of protein can be obtained and efficient purification and refolding strategies are available. Thus, selection of appropriate expression systems (including vectors and cell types) is within the knowledge of one skilled in the art. Similarly, once the primary amino acid sequence for the fusion protein of the present invention is chosen, one of ordinary skill in the art can easily design appropriate recombinant DNA constructs which will encode the desired amino acid sequence, taking into consideration such factors as codon biases in the chosen host, the need for secretion signal sequences in the host, the introduction of proteinase cleavage sites within the signal sequence, and the like.

Expression of the MBP/DC-SIGN fusion protein of the present invention may conveniently be achieved by culturing under appropriate conditions recombinant host cells containing the nucleic acid. Thus, it is well within the abilities and discretion of the skilled artisan, without undue experimentation, to choose an appropriate or optimal expression system. Similarly, once the primary amino acid sequence for the polypeptide of the present invention is chosen, one of ordinary skill in the art can easily design appropriate polynucleotides such as recombinant DNA constructs which will encode the desired proteins, taking into consideration such factors as codon biases in the chosen host, the need for secretion signal sequences in the host, the introduction of proteinase cleavage sites within the signal sequence, and the like. These recombinant DNA constructs may be inserted in-frame into any of a number of expression vectors appropriate to the chosen host. The choice of an appropriate or optimal expression vector is, again, a matter well within the ability and discretion of the skilled practitioner. In certain embodiments, the expression vector will include a strong promoter to drive expression of the recombinant constructs.

In an embodiment, the MBP/DC-SIGN fusion protein of the invention can be isolated using suitable standard procedures well known in the art, and optionally subjected to further processing such as, for example, lyophilization.

In one embodiment the isolated polynucleotide encodes a polypeptide comprising the fusion protein. In an embodiment the isolated polynucleotide encodes an MBL polypeptide with effector function and a second polypeptide that includes a targeting sequence. In certain embodiments, the polypeptides are encoded in a single contiguous polynucleotide sequence (a genetic fusion). In other embodiments, polypeptides are encoded by non-contiguous polynucleotide sequences. Accordingly, in some embodiments the polypeptides expressed, isolated, and purified as separate polypeptides and fused together to form the fusion protein of the invention.

These recombinant DNA constructs may be inserted in-frame into any of a number of expression vectors appropriate to the chosen host. In certain embodiments, the expression vector comprises a strong promoter that controls expression of the recombinant fusion protein constructs. When recombinant expression strategies are used to generate the fusion protein of the invention, the resulting fusion protein can be isolated and purified using suitable standard procedures well known in the art, and optionally subjected to further processing such as, for example, lyophilization.

Standard techniques may be used for recombinant DNA molecule, protein, and fusion protein production, as well as for tissue culture and cell transformation. See, e.g., Sambrook, et al. (below) or Current Protocols in Molecular Biology [Ausubel et al., eds., Green Publishers Inc. and Wiley and Sons 1994]. Purification techniques are typically performed according to the manufacturer's specifications or as commonly accomplished in the art using conventional procedures such as those set forth in Sambrook et al. [Molecular Cloning: A Laboratory Manual. Cold Spring Harbor Laboratory Press, Cold Spring Harbor, N.Y. (1989], or as described herein. Unless specific definitions are provided, the nomenclature utilized in connection with the laboratory procedures, and techniques relating to molecular biology, biochemistry, analytical chemistry, and pharmaceutical/formulation chemistry described herein are those well known and commonly used in the art. Standard techniques can be used for biochemical syntheses, biochemical analyses, pharmaceutical preparation, formulation, and delivery, and treatment of patients.

It will be appreciated that a flexible molecular linker optionally can be interposed between, and covalently join, the first and second polypeptides of the fusion protein. In certain embodiments, the linker comprises a polypeptide sequence of about 1-20 amino acid residues. The linker may be less than 10 amino acids, most preferably, 5, 4, 3, 2, or 1. It may be in certain cases that 9, 8, 7 or 6 amino acids are suitable. In some embodiments the linker is essentially non-immunogenic, not prone to proteolytic cleavage and does not comprise amino acid residues which are known to interact with other residues (e.g. cysteine residues).

The description below also relates to methods of producing fusion proteins and trimeric complexes that are covalently attached (hereinafter “conjugated”) to one or more chemical groups. Chemical groups suitable for use in such conjugates are preferably not significantly toxic or immunogenic. The chemical group is optionally selected to produce a conjugate that can be stored and used under conditions suitable for storage. A variety of exemplary chemical groups that can be conjugated to polypeptides are known in the art and include for example carbohydrates, such as those carbohydrates that occur naturally on glycoproteins, polyglutamate, and non-proteinaceous polymers, such as polyols (see, e.g., U.S. Pat. No. 6,245,901).

A polyol, for example, can be conjugated to fusion proteins of the invention at one or more amino acid residues, including lysine residues, as is disclosed in WO 93/00109. The polyol employed can be any water-soluble poly(alkylene oxide) polymer and can have a linear or branched chain. Suitable polyols include those substituted at one or more hydroxyl positions with a chemical group, such as an alkyl group having between one and four carbons. Typically, the polyol is a poly(alkylene glycol), such as poly(ethylene glycol) (PEG), and thus, for ease of description, the remainder of the discussion relates to an exemplary embodiment wherein the polyol employed is PEG and the process of conjugating the polyol to a polypeptide is termed “pegylation.” However, those skilled in the art recognize that other polyols, such as, for example, polypropylene glycol) and polyethylene-polypropylene glycol copolymers, can be employed using the techniques for conjugation described herein for PEG.

The average molecular weight of the PEG employed in the pegylation can vary, and typically ranges from about 500 to about 30,000 daltons (D). Preferably, the average molecular weight of the PEG is from about 1,000 to about 25,000 D, and more preferably from about 1,000 to about 5,000 D. In one embodiment, pegylation is carried out with PEG having an average molecular weight of about 1,000 D. Optionally, the PEG homopolymer is unsubstituted, but it may also be substituted at one end with an alkyl group. Preferably, the alkyl group is a C1-C4 alkyl group, and most preferably a methyl group. PEG preparations are commercially available, and typically, those PEG preparations suitable for use in the present invention are nonhomogeneous preparations sold according to average molecular weight. For example, commercially available PEG(5000) preparations typically contain molecules that vary slightly in molecular weight, usually ±500 D. The fusion protein of the invention can be further modified using techniques known in the art, such as, conjugated to a small molecule compounds (e.g., a chemotherapeutic); conjugated to a signal molecule (e.g., a fluorophore); conjugated to a molecule of a specific binding pair (e.g. biotin/streptavidin, antibody/antigen); or stabilized by glycosylation, PEGylation, or further fusions to a stabilizing domain (e.g., Fc domains).

A variety of methods for pegylating proteins are known in the art. Specific methods of producing proteins conjugated to PEG include the methods described in U.S. Pat. Nos. 4,179,337, 4,935,465 and 5,849,535. Typically the protein is covalently bonded via one or more of the amino acid residues of the protein to a terminal reactive group on the polymer, depending mainly on the reaction conditions, the molecular weight of the polymer, etc. The polymer with the reactive group(s) is designated herein as activated polymer. The reactive group selectively reacts with free amino or other reactive groups on the protein. The PEG polymer can be coupled to the amino or other reactive group on the protein in either a random or a site specific manner. It will be understood, however, that the type and amount of the reactive group chosen, as well as the type of polymer employed, to obtain optimum results, will depend on the particular protein or protein variant employed to avoid having the reactive group react with too many particularly active groups on the protein. As this may not be possible to avoid completely, it is recommended that generally from about 0.1 to 1000 moles, preferably 2 to 200 moles, of activated polymer per mole of protein, depending on protein concentration, is employed. The final amount of activated polymer per mole of protein is a balance to maintain optimum activity, while at the same time optimizing, if possible, the circulatory half-life of the protein.

Furthermore, other half-life extending molecules can be attached to the N- or C-terminus of the MBL polypeptide including serum albumin-binding peptides, IgG-binding peptides or peptides binding to FcRn.

It should be noted that the section headings are used herein for organizational purposes only, and are not to be construed as in any way limiting the subject matter described. All references cited herein are incorporated by reference in their entirety for all purposes.

The Examples that follow are merely illustrative of certain embodiments of the invention, and are not to be taken as limiting the invention, which is defined by the appended claims.

### EXAMPLES

### Example 1

### Construction and Expression in HEK293 Cells of Tagged and Untagged MBP-CD 209 CTLD Fusion Protein Expression Plasmid Clones

DC-SIGN (Dendritic cell specific ICAM-3 grabbing nonintegrin) is a type II transmembrane protein belonging to the C-type lectin family. The protein is expressed on the surface of dendritic cells (DC) in the periphery and participates in the primary contact between the antigen-presenting cells and resting T-cells in the lymphatic system via ICAM-3 on the T-cells. DC-SIGN also interacts with ICAM-2 on epithelial cells during migration of DCs to lymphoid tissues. DC-SIGN also binds strongly to the HIV envelope protein gp120 and facilitates viral infection in trans of CD4+ T-cells. The DC-SIGN protein consists of a short amino-terminal cytoplasmic tail, a transmembrane domain, a stalk of up to 7½ repeats, followed by a C-terminal C-type carbohydrate recognition domain (CRD). The stalk promotes the formation of tetramers (coiled coil). DC-SIGN binds to mannose- and fucose-containing complex glycoconjugates including the Lewis antigens Le^{x} and Le^{y}. In contrast to most other lectins, DC-SIGN binds to internal parts of the carbohydrate structure, presumably increasing the possibility of this scaffold to obtain more diverse and specific carbohydrate binding.

A series of fusion constructs representing the MBP signal sequence, followed by the Cystenyl rich oligomerisation domain, the collagenous repeat region, various derivatives representing fusions of the MBP neck region and DC-SIGN CTLD domain. The nucleotide sequence of the inserts encoding the different MBP/DC-SIGN CTLD fusion proteins (MBP/DC-SIGN CTLD-ABs (SEQ ID NO: 1), MBP/DC-SIGN CTLD-ACs (SEQ ID NO: 3), MBP/DC-SIGN CTLD-ADs (SEQ ID NO: 5), MBP/DC-SIGN CTLD-ABsC (SEQ ID NO: 7), MBP/DC-SIGN CTLD-ACsC (SEQ ID NO: 9), MBP/DC-SIGN CTLD-ADsC (SEQ ID NO: 11), MBP/DC-SIGN CTLD-FE (SEQ ID NO: 13), MBP/DC-SIGN CTLD-GE (SEQ ID NO: 15), MBP/DC-SIGN CTLD-HE (SEQ ID NO: 17), MBP/DC-SIGN CTLD-ACsCSG (SEQ ID NO: 19), MBP/DC-SIGN CTLD-ACsCSGGS (SEQ ID NO: 21), and MBP/DC-SIGN CTLD-ACsCSGGGS (SEQ ID NO:23) MBP/DC-SIGN CTLD-ABs0 (SEQ ID NO:25), and MBP/DC-SIGN CTLD-ABsC0 (SEQ ID NO:27), respectively were cloned using a series of plasmid and insert specific oligonucleotide primers, as shown in Table 1. MBP/DC-SIGN CTLD-ABs, MBP/DC-SIGN CTLD-ACs, and MBP/DC-SIGN CTLD-ADs contain C-terminally truncated DC-SIGN CTLD domains; MBP/DC-SIGN CTLD-ABsC, MBP/DC-SIGN CTLD-ACsC, and MBP/DC-SIGN CTLD-ADsC contain full-length DC-SIGN CTLD domains; MBP/DC-SIGN CTLD-FE, MBP/DC-SIGN CTLD-GE, and MBP/DC-SIGN CTLD-HE contain N- and C-terminal DC-SIGN CTLD domains; and MBP/DC-SIGN CTLD-ACsCSG, MBP/DC-SIGN CTLD-ACsCSGGS, and MBP/DC-SIGN CTLD-ACsCSGGGS contain Serine-Glycine insertions at the N-terminus of the DC-SIGN CTLD domains. The constructs were then verified using the GENETIC 3700 analyzer and the sequencing BIG DYE® ver. 3.0 sequencing (Applied Biosystems).

TABLE 1

| | 3′ | 5′ |
|---|---|---|
| | Oligonuclotide | Oligonucleotide |
| pMBP/DC-SIGN CTLD-ABs | SEQ ID NO: 29 | SEQ ID NO: 30 |
| MBP/DC-SIGN CTLD-ACs | SEQ ID NO: 29 | SEQ ID NO: 31 |
| MBP/DC-SIGN CTLD-ADs | SEQ ID NO: 29 | SEQ ID NO: 32 |
| MBP/DC-SIGN CTLD-ABsC | SEQ ID NO: 29 | SEQ ID NO: 30 |
| MBP/DC-SIGN CTLD-ACsC | SEQ ID NO: 29 | SEQ ID NO: 32 |
| MBP/DC-SIGN CTLD-ADsC | SEQ ID NO: 29 | SEQ ID NO: 32 |
| MBP/DC-SIGN CTLD-FE | SEQ ID NO: 34 | SEQ ID NO: 33 |
| MBP/DC-SIGN CTLD-GE | SEQ ID NO: 35 | SEQ ID NO: 33 |
| MBP/DC-SIGN CTLD-HE | SEQ ID NO: 36 | SEQ ID NO: 33 |
| MBP/DC-SIGN CTLD-ACsCSG | SEQ ID NO: 37 | SEQ ID NO: 31 |
| MBP/DC-SIGN CTLD- | SEQ ID NO: 37 | SEQ ID NO: 31 |
| ACsCSGGS | | |
| MBP/DC-SIGN CTLD- | SEQ ID NO: 37 | SEQ ID NO: 31 |
| ACsCSGGGS | | |
| MBP/DC-SIGN CTLD-ABs0 | SEQ ID NO: 29 | SEQ ID NO: 30 |
| MBP/DC-SIGN CTLD-ABsC0 | SEQ ID NO: 29 | SEQ ID NO: 30 |

The clones capable of expressing the fusion proteins MBP/DC-SIGN CTLD-ABs (SEQ ID NO: 2), MBP/DC-SIGN CTLD-ACs (SEQ ID NO: 4), MBP/DC-SIGN CTLD-ADs (SEQ ID NO: 6), MBP/DC-SIGN CTLD-ABsC (SEQ ID NO: 8), MBP/DC-SIGN CTLD-ACsC (SEQ ID NO:10), MBP/DC-SIGN CTLD-ADsC (SEQ ID NO:12), MBP/DC-SIGN CTLD-FE (SEQ ID NO:14), MBP/DC-SIGN CTLD-GE (SEQ ID NO:16), and MBP/DC-SIGN CTLD-HE SEQ ID NO:18), MBP/DC-SIGN CTLD-ACsCSG (SEQ ID NO:20), MBP/DC-SIGN CTLD-ACsCSGGS (SEQ ID NO:22), and MBP/DC-SIGN CTLD-ACsCSGGGS (SEQ ID NO:24) all tagged with a myc- and His tags on their amino terminal ends, and the two untagged fusion protein derivatives MBP/DC-SIGN CTLD-ABs0 (SEQ ID NO:26) and MBP/DC-SIGN CTLD-ABsC0 (SEQ ID NO:28), were constructed using a sequential oligonucleotide assembly and subcloning strategy into the commercially available expression plasmid pcDNA3.1 (Invitrogen) yielding the resulting plasmids: pMBP/DC-SIGN CTLD-ABs, pMBP/DC-SIGN CTLD-ACs, pMBP/DC-SIGN CTLD-ADs, pMBP/DC-SIGN CTLD-ABsC, pMBP/DC-SIGN CTLD-ACsC, pMBP/DC-SIGN CTLD-ADsC, pMBP/DC-SIGN CTLD-FE, pMBP/DC-SIGN CTLD-GE, pMBP/DC-SIGN CTLD-HE, pMBP/DC-SIGN CTLD-ACsCSG, pMBP/DC-SIGN CTLD-ACsCSGGS, pMBP/DC-SIGN CTLD-ACsCSGGGS, pMBP/DC-SIGN CTLD-ABs0, and MBP/DC-SIGN CTLD-ABsC0. Plasmids were transformed into E. coli XL-1 Blue cells (Stratagene) for plasmid propagation and nucleotide sequence verification of the inserts.

Plasmid DNA for transfection into human embryonic kidney cells (HEK293 cells) was isolated using the Qiagen MAXI PREP® maxi prep procedure. Initially, only the tagged MBP/DC-SIGN CTLD fusion protein derivatives were transfected and analyzed for expression. Cells were transfected using the lipofectamine protocol (Invitrogen). All constructs were successfully transiently transfected and culture supernatants were analyzed after four days for ability to bind immobilized Lewis tumour antigen y (Le^{y}) coupled to human serum albumin (HSA) or the Le^{y} expressing human breast cancer cell line SKBR-3.

### Example 2

### Analysis of the Various Tagged MBP-CD209 CTLD Fusion Proteins Binding to Either Immobilised HSA-Le^{y} or Le^{y} Expressing Cells of the Human Breast Cancer Cell Line SKBR-3

Supernatants from HEK293 cell culture transfected with tagged MBP/DC-SIGN CTLD expressing plasmids (pMBP/DC-SIGN CTLD-ABs, pMBP/DC-SIGN CTLD-ACs, pMBP/DC-SIGN CTLD-ADs, pMBP/DC-SIGN CTLD-ABsC, pMBP/DC-SIGN CTLD-ACsC, pMBP/DC-SIGN CTLD-ADsC, pMBP/DC-SIGN CTLD-FE, pMBP/DC-SIGN CTLD-GE, and pMBP/DC-SIGN CTLD-HE) were analyzed for ability to bind to immobilized Le^{y} coupled to human serum albumin (HSA) or to Le^{y} expressing human SKBR-3 breast cancer cells after four days of transient expression. The MBP/DC-SIGN CTLD ABs and MBP/DC-SIGN CTLD ABsC fusion proteins were also tested for ability to bind to MCF-7 human breast cancer cells, LNCap prostate cancer cells, and A431 skin epithelial squamous carcinoma cells.

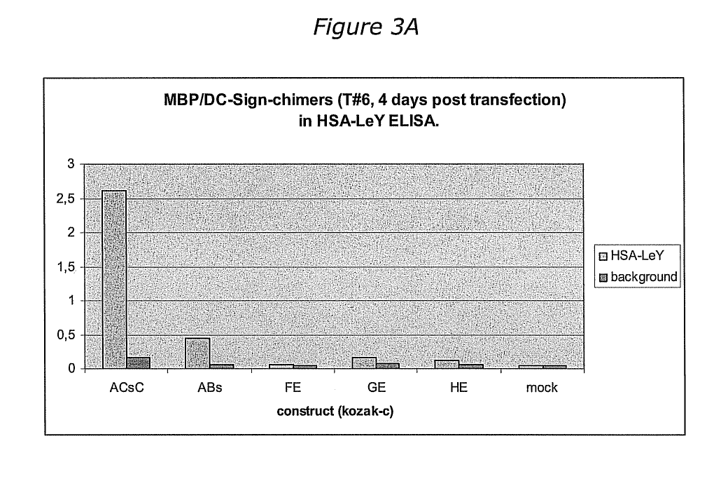

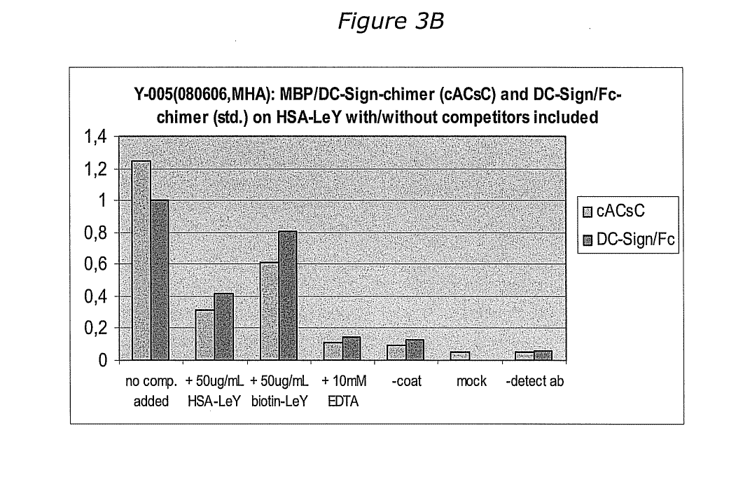

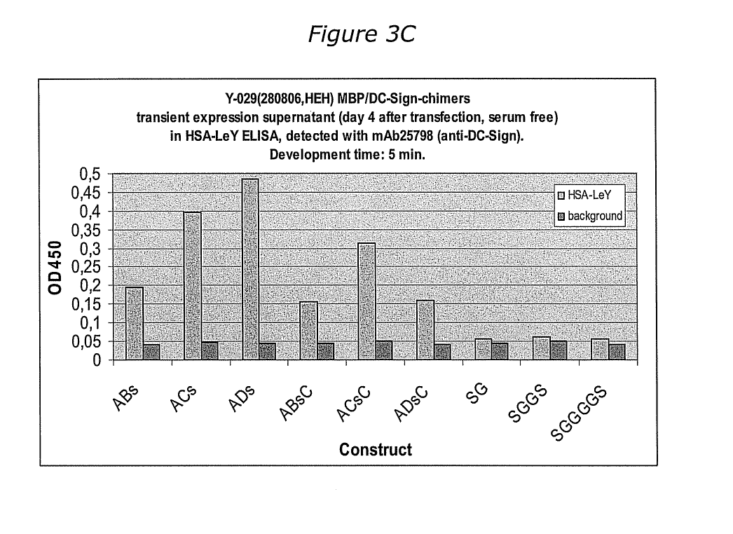

In the first assay 0.5 μg Le^{y}-HSA (IsoSep, Uppsala Sweden) per well in PBS (10 mM sodium phosphate pH 7.4, 100 mM NaCl) were incubated overnight and immobilized in a 96 well ELISA tray. After washing away unbound Le^{y}-HSA and blocking, 100 μl of each of the culture supernatants were analyzed for binding in an ELISA assay (FIGS. 3A, 3B, and 3C). A commercially available DC-SIGN CTLD Fc fusion protein (R&D systems) was used as a positive control and the anti-DC-SIGN mouse monoclonal antibody clone MR-1 (Abcam) was use for detection, followed by a HRP-conjugated anti-mouse IgG antibody.

The fusion proteins MBP/DC-SIGN CTLD ACs, MBP/DC-SIGN CTLD ACsC, and MBP/DC-SIGN CTLD ADs showed the strongest binding, MBP/DC-SIGN CTLD ABs, MBP/DC-SIGN CTLD ABsC, and MBP/DC-SIGN CTLD ADsC showed intermediate binding, and the remaining fusion protein showed no binding (FIG. 3A, 3C). The binding of the MBP/DC-SIGN CTLD ACsC fusion protein was compared to the binding of a commercially available DC-SIGN CTLD Fc compound (FIG. 3B). Binding was found to be specific and calcium dependent.

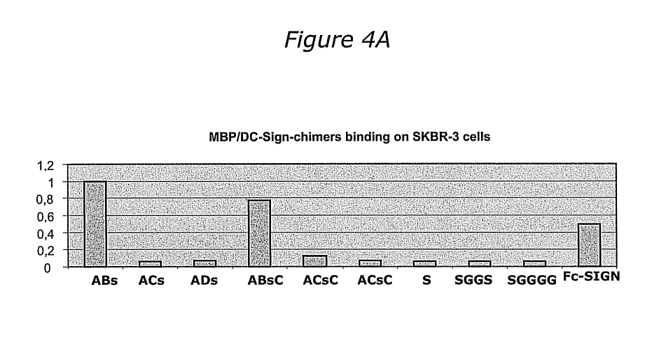

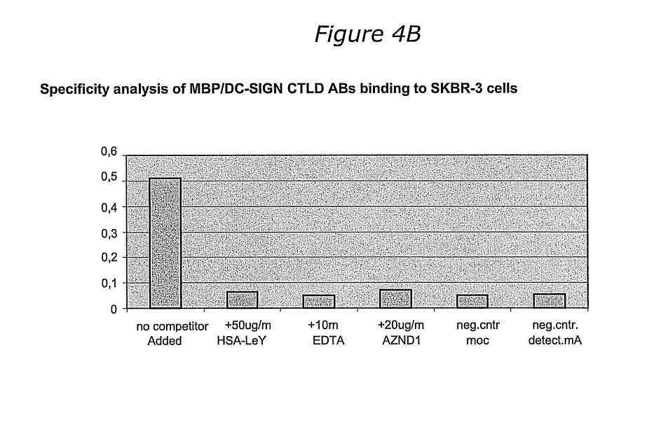

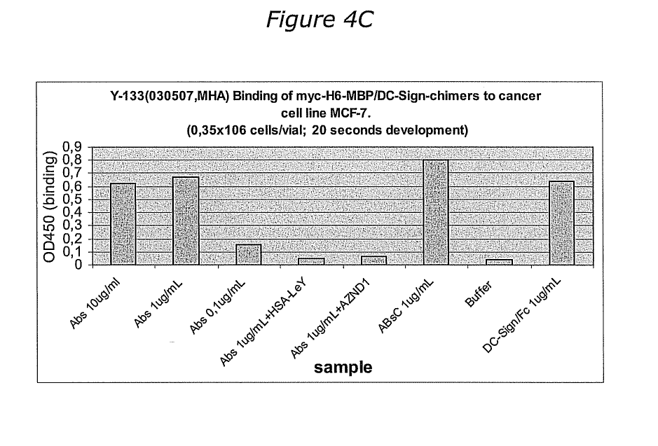

In an additional assay, semi-confluent cultures of Le^{y} expressing SKBR-3 or MCF-7 cells grown at 37° C. and 5% CO2 in McCoy or DMEM medium, respectively, supplemented with 10% fetal calf serum, and 1% Pen/Strep, were scraped off the plastic surface, washed and blocked with 1% BSA and incubated for one hour with culture supernatant expressing each of the MBP-DC-SIGN CTLD fusion proteins or purified fusion protein. Following careful washing, the amount of bound fusion protein was determined in a suspension phase ELISA assay (FIGS. 4A-C). The commercially available fusion protein DC-SIGN/Fc (R&D systems) was included as a positive control. The anti-DC-SIGN mouse monoclonal antibody clone MR-1 (Abcam) was use for detection, followed by a HRP-conjugated anti-mouse IgG antibody.

With respect to the LNCap and A431 cancer cell lines the cells were grown to 70% confluence in Nunclon 96-trays in respectively RPMI and DMEM medium containing 10% FBS and 1% Pen/Strep. After washing and blocking of the cells they were incubated for one hour with the purified MBP-DC-SIGN CTLD fusion proteins. Following careful washing the amount of bound fusion protein was determined in an ELISA assay (FIG. 4A-C). The commercially available fusion protein DC-SIGN/Fc (R&D Systems) was included as a positive control. The anti-DC-SIGN mouse monoclonal antibody clone MR-1 (Abcam) was use for detection, followed by a HRP-conjugated anti-mouse IgG antibody.

The fusion proteins MBP/DC-SIGN CTLD ABs and MBP/DC-SIGN CTLD ABsC showed strongest binding to the SKBR-3 cells (FIG. 4A) and binding was demonstrated to be specific for Le^{y} and calcium dependent (FIG. 4B). Evaluation of the fusion proteins MBP/DC-SIGN CTLD ABs and MBP/DC-SIGN CTLD ABsC binding to MCF-7 cells is shown in FIG. 4C.

### Example 3

### Purification of MBP/DC-SIGN CTLD Derivatives Using Mannan-Agarose Affinity Chromatography

Stable clonal cell lines expressing MBP/DC-SIGN CTLD ABs, MBP/DC-SIGN CTLD ABsC, or MBP/DC-SIGN CTLD ABsC0 fusion derivatives and a stable cell line population expressing the MBP/DC-SIGN CTLD ABs0 fusion derivative were established by transfection of HEK293 cells with either super coiled or linearized plasmid DNA, and different concentrations of plasmid DNA. Stable cell lines were obtained by seeding cells at various concentrations and increasing selection pressure using zeocin. Several clones were propagated and the culture supernatant were analysed for fusion protein production using the immobilised Le^{y} HSA ELISA assay described in Example 2. MBP/DC-SIGN CTLD derivatives from the supernatant of stably transfected clones were affinity purified using mannan-sepharose as described in the following paragraph.

The MBP/DC-SIGN CTLD expression supernatant (ca. 2.5 L) was filtered, supplied with 250 mL of 10×TBSC-buffer (1×TBSC: 10 mM Tris-HCl pH 7.5, 150 mM NaCl, 2 mM Ca²⁺) and applied at 0.5 mL/min to a 25 mL mannan-agarose column (Sigma) at 4° C. After application, the column was washed with 2 column volumes of 1×TBSC and eluted with 1×TBS, 5 mM EDTA. Calcium chloride was added to the eluted protein fractions to 5 mM and dialysed against 500 volumes of 1×TBSC-buffer. The protein concentrations were determined by spectroscopy (A₂₈₀), and the purity verified by SDS-PAGE analysis.

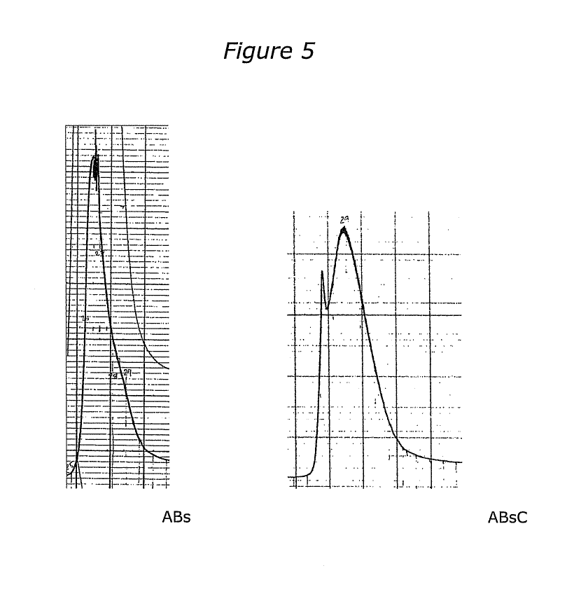

The elution profiles of the MBP/DC-SIGN CTLD ABs and −ABsC were not similar (FIG. 5). The MBP/DC-SIGN CTLD ABs elutes as one sharp peak whereas ABsC elutes as two peaks, the first one smaller than the last one. Both elution profiles have a sharp front and a longer tail.

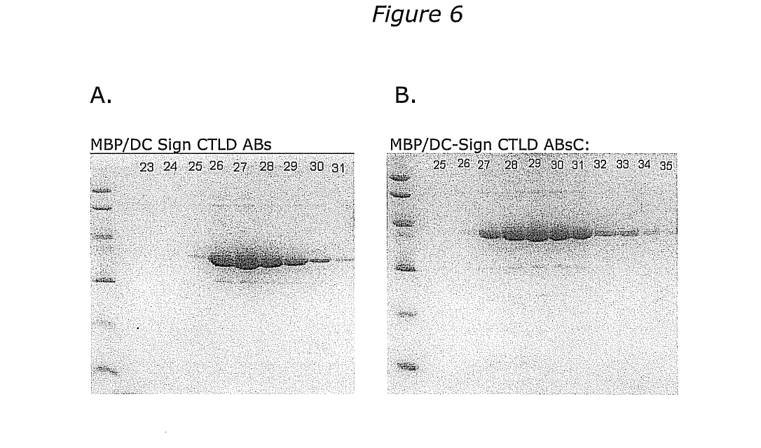

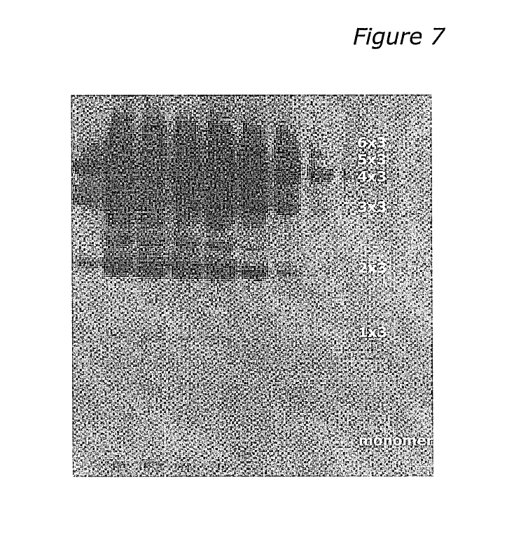

2 mg of each derivative were isolated from 2.5 L of culture supernatant at a purity of >90% as judged by SDS-PAGE analysis (FIG. 6A-B). The concentration of the fusion protein was in each case 300-660 μg/mL in the peak fractions. The oligomerization profile of the isolated derivatives was analyzed (FIG. 7) on Western Blots of non-reduced samples separated by 3-8% gradient SDS-PAGE using the DC-SIGN specific mouse monoclonal antibody clone MR-1 (Abcam).

### Example 4

### Analysis of MBP/DC-SIGN CTLD ABs and −ABsC Derivatives Binding to Immobilised Le^{y}-HSA Compared to the Binding of a Commercially Available (DC-SIGN)₂-Fc Derivative

An ELISA assay with Le^{y}-coupled HSA immobilized in wells in a microtiter plate (Nunc) was develop to analyze the strength of binding of the MBP/DC-SIGN CTLD ABs and −ABsC derivatives compared to the divalent DC-SIGN Fc derivative from R&D systems.

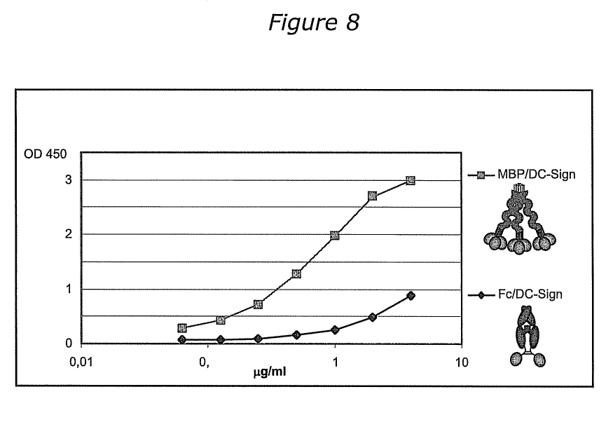

In each well of a 96 well ELISA tray was added 0.5 mg Le^{y}-HSA (IsoSep) in PBS (10 mM sodium phosphate pH 7.4, 100 mM NaCl) which was incubated overnight and immobilized. After washing away unbound Le^{y}-HSA and blocking, serial dilutions of MBP/DC-SIGN CTLD ABs, −ABcC, or (DC-SIGN)₂-Fc in one hundred microliters of 1×TBSC were added to each well and analysed for binding in the ELISA assay. An anti-DC-SIGN mouse monoclonal antibody clone MR-1 (Abcam) was used for detection, followed by a HRP-conjugated anti-mouse IgG antibody. A typical result from the comparative analysis is illustrated in FIG. 8, which demonstrates the increased binding of the MBP/DC-SIGN CTLD fusions (squares) relative to the Fc/DC-SIGN fusion (diamonds).

### Example 5

### Purification of MBP/DC-SIGN CTLD Derivatives Using D-Mannose Sepharose Affinity Chromatography

MBP/DC-SIGN-CTLD derivatives were isolated from supernatants of stably transfected clones via affinity purification on a D-mannose-sepharose matrix followed by further purification (a “polishing step”) by ionexchange chromatography on a Source 15Q column.

The MBP/DC-SIGN CTLD expression supernatant was supplied with 10×TBSC-buffer to 1×TBSC final (10 mM Tris-HCl pH 7.5, 150 mM NaCl, 2 mM Ca²⁺) and run over a D-mannose-sepharose column at 4° C. The D-mannose-sepharose matrix was prepared by coupling D-mannose to sepharose 6-BCl activated by di-vinyl sulphone following a standard protocol. After application, the column was washed with two column volumes of 1×TBSC and eluted with 1×TBS, 5 mM EDTA. The eluted protein fractions were added CaCl₂ to 5 mM and dialyzed against 5-10 volumes of 1×TBSC-buffer. After elution, inactivation of potential virus was achieved by adding Tween 80 to 1% w/v and tri(n-butyl)phosphate to 0.3% w/v and leaving the eluted material for 6 hrs at room temperature. After clarification by centrifugation, the material was loaded onto the Source 15Q column in 1×TBSC. Once loaded the column was flushed with five column volumes of 15 mM Na₂HPO₄ pH 8.0, 25 mM NaCl. Column was eluted over gradient with 15 mM Na₂HPO₄ pH 8.0, 25 mM NaCl. The eluted protein was diafiltrated into 10 mM NaPO₄ pH 7.5, 100 mM NaCl. The protein purity was then analyzed by SDS-PAGE and concentrations were determined by spectroscopy (A₂₈₀).

### Example 6

### Analysis of Initiation of Complement Lysis on Immobilised Le^{y}-HSA as Monitored by C4 Cleavage

The purified MBP/DC-SIGN CTLD ABs and −ABsC derivatives produced either as described in Examples 3 and 5 have been assayed in a complement activation assay. This assay is a quantitative measurement of the ability of MBL/DC-SIGN CTLD/MASP complexes to initiate C4 cleavage when bound to HSA-LeY. The deposited C4-fragments were then quantitated by an anti-C4 antibody.

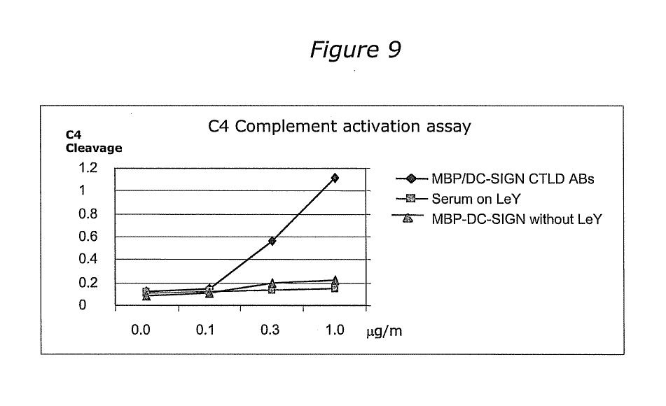

Microtiterplates were coated overnight with 5 μg/mL HSA-LeY in PBS. After washing away excessive antigen the plates are blocked with 0.1% BSA in TBS (10 mM Tris-HCl; 140 mM NaCl; pH7.4). MBP/DC-SIGN CTLDs are complexed with 2% MBL deficient human serum (State Serum Institute, Copenhagen, Denmark) in MBL binding buffer (20 mM Tris-HCl; 10 mM CaCl₂; 1M NaCl, 0.05% TritonX-100; 0.1% BSA; pH7.4) and allowed to bind overnight at 4° C. The plates were temperated and washed. 5 μg/mL human C4 protein (Quidel) is added to the wells which were incubated for 1.5 hours. Wells were washed and the cleaved C4 fragments detected with a polyclonal anti-C4 antibody (DAKO) followed by a HRP-conjugated anti-rabbit Ig antibody (DAKO) in an ELISA assay (FIG. 9).

### Example 7

### Analysis of Initiation of Complement Lysis of Epithelial Cancer Cells as Monitored by C4 Cleavage

The purified MBP/DC-SIGN CTLD ABs and −ABsC derivatives produced either as described in Example 3 or 5 were assayed in a standard complement activation assay. This assay is a quantitative measurement of the ability of MBL/DC-SIGN CTLD/MASP complexes to initiate C4 cleavage when bound to LeY tumour antigen on epithelial cancer cells. The deposited C4-fragments are then quantitated by an anti-C4 antibody.

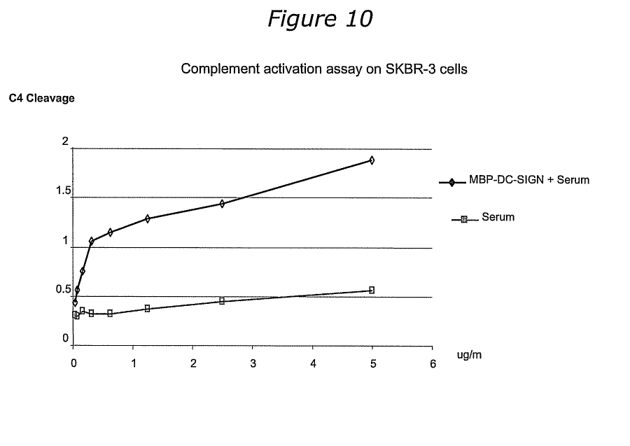

Cells (e.g. SKBR-3, MCF-7 and others) were grown to 70% confluence and scraped off the plastic. After washing and preblocking in 0.5% BSA/TBSC-buffer the cells were resuspended in a buffer containing MBP/DC-SIGN CTLD complexed with 2% MBL deficient human serum (State Serum Institute, Copenhagen, Denmark) in 0.5% BSA/1×TBSC buffer and allowed to bind for 2 hours at RT. The cells were washed in 0.5% BSA/TBSC-buffer and resuspended in human C4 (5 μg/mL in 0.5% BSA/TBSC) and incubated at RT for one hour. Cells were again washed and the cleaved C4 fragments were detected with a polyclonal anti-C4 antibody (DAKO) followed by a HRP-conjugated anti-rabbit Ig antibody (DAKO) in a suspension ELISA assay (FIG. 10).

### Example 8

### Analysis of Inhibition of Epithelial Cancer Cell Proliferation by MBP/DC-SIGN CTLD Derivatives or a Monoclonal Antibody

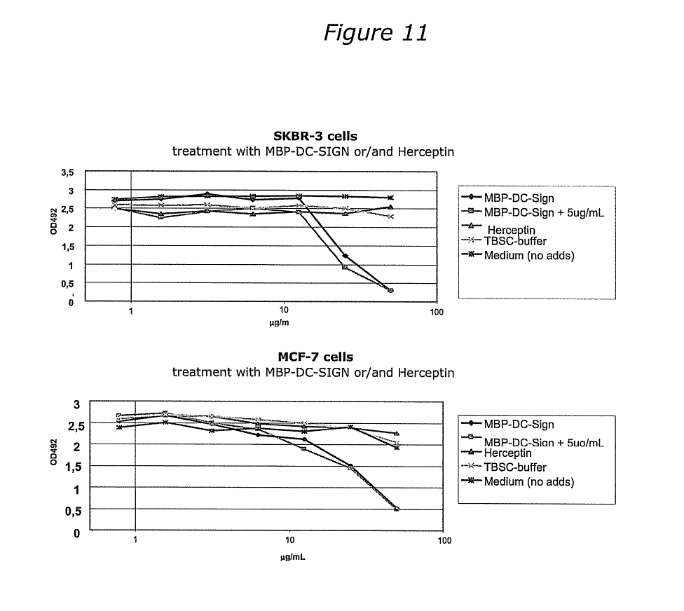

The breast cancer cell lines SKBR-3 and MCF-7 were seeded in Nunclon 96-trays at 5000 cells/well in respectively McCoy and DMEM medium containing 10% FBS and 1% Pen/Strep at 37° C. and 5% CO₂ overnight. Dilutions of MBP/DC-SIGN-ABs, MBP/DC-SIGN-ABsC0, MBP/DC-SIGN-ABsC0 (+5 μg/mL Herceptin), Herceptin, and a buffer control were added to the cells and cells were allowed to grow at 37° C. and 5% CO₂ for two or five days. The MBP/DC-SIGN derivatives were isolated using the protocol described in Example 5. Hereafter the number of viable cells was measured using a colorimetric assay (CellTiter 96 AQueous Non-Radioactive Cell Proliferation Assay) according to the manufacturer's instructions (Promega). The result of the assay after five days is illustrated in FIG. 11.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in molecular biology, immunology, chemistry, biochemistry or in the relevant fields are intended to be within the scope of the appended claims.

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention.

The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Any numerical values recited herein include all values from the lower value to the upper value in increments of one unit provided that there is a separation of at least two units between any lower value and any higher value. As an example, if it is stated that the concentration of a component or value of a process variable such as, for example, size, angle size, pressure, time and the like, is, for example, from 1 to 90, specifically from 20 to 80, more specifically from 30 to 70, it is intended that values such as 15 to 85, 22 to 68, 43 to 51, to 32, etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Particular methods, devices, and materials are described, although any methods and materials similar or equivalent to those described herein can be used in the practice or testing of the invention. The disclosures of all references and publications cited herein are expressly incorporated by reference in their entireties to the same extent as if each were incorporated by reference individually.

What is claimed is:

1. A fusion protein comprising a first polypeptide comprising a mannose binding lectin (MBL) polypeptide having effector function and a second polypeptide comprising a targeting sequence that binds to a cell surface or to a virus, wherein the first polypeptide does not comprise an active MBL C-Type Lectin Like Domain (CLTD).

2. The fusion protein of claim 1 wherein the targeting sequence binds to a receptor on the surface of a cell selected from the group consisting of tumor cells, immune cells, bacterial cells, protozoa, fungi and a cell infected with a virus.

3. The fusion protein of claim 2, wherein the immune cells are selected from inflammatory immune cells and suppressive immune cells.

4. The fusion protein of claim 1 wherein the targeting molecule is a lectin.

5. The fusion protein of claim 1 wherein the lectin is Dendritic Cell specific ICAM-3 grabbing nonintegrin (DC-SIGN).

6. The fusion protein of claim 1 wherein the first polypeptide comprises SEQ ID NO: 49.

7. The fusion protein of claim 1, wherein the first polypeptide binds to MBP-associated serine proteases (MASP).

8. The fusion protein of claim 1, wherein the protein activates a mammalian complement system.

9. The fusion protein of claim 1, wherein the second polypeptide comprises a CTLD having a loop region comprising the targeting sequence, wherein the CTLD is not an MBP CTLD.

10. A fusion protein of claim 1 selected from the group consisting of MBP/DC-SIGN CTLD-ABs (SEQ ID NO: 2), MBP/DC-SIGN CTLD-ACs (SEQ ID NO: 4), MBP/DC-SIGN CTLD-ADs (SEQ ID NO: 6), MBP/DC-SIGN CTLD-ABsC (SEQ ID NO: 8), MBP/DC-SIGN CTLD-ACsC (SEQ ID NO:10), MBP/DC-SIGN CTLD-ADsC (SEQ ID NO:12), MBP/DC-SIGN CTLD-FE (SEQ ID NO:14), MBP/DC-SIGN CTLD-GE (SEQ ID NO:16), and MBP/DC-SIGN CTLD-HE SEQ ID NO:18), MBP/DC-SIGN CTLD-ACsCSG (SEQ ID NO:20), MBP/DC-SIGN CTLD-ACsCSGGS (SEQ ID NO:22), and MBP/DC-SIGN CTLD-ACsCSGGGS (SEQ ID NO:24), MBP/DC-SIGN CTLD-ABs0 (SEQ ID NO:26) and MBP/DC-SIGN CTLD-ABsC0 (SEQ ID NO:28).

11. A method of activating a mammalian complement system comprising administering to the mammal the fusion protein of claim 1.

12. A pharmaceutical composition comprising the fusion protein of claim 1 and a pharmaceutically acceptable excipient.

13. A pharmaceutical composition of claim 12, further comprising at least one of a chemotherapeutic agent and a therapeutic agent.

14. The pharmaceutical composition of claim 13, wherein the at least one therapeutic agent comprises at least one of an antibody, a kinase inhibitor, or a cancer vaccine.

15. A pharmaceutical composition according to claim 11, wherein the chemotherapeutic agent is selected from raltitrexed, doxorubicin, taxol, 5-fluorouracil, irinotecan and cisplatin, mitomycin-C, and oxaliplatin, and the therapeutic agent is trastuzumab.

16. A method of treating a pathogenic disease comprising administering to a patient suffering from the disease and effective amount of the pharmaceutical composition of claim 11 wherein the targeting sequence binds to a cell surface marker of the pathogen or a marker on a cell that is infected with a virus.

17. A method of treating a proliferative disease comprising tumor cells comprising administering to a patient in need thereof an effective amount of the pharmaceutical composition of claim 11 wherein the targeting sequence binds to a marker on the surface of the tumor cells.

18. The method of claim 15, further comprising administering to the patient a cancer vaccine.

19. The method of claim 15, wherein the receptor comprises a Lewis antigen.

20. The method of claim 17, wherein the targeting sequence comprises a DC-SIGN polypeptide sequence that binds to a Lewis antigen.

21. A method of treating cancer in a subject comprising administering to said subject an effective amount of the pharmaceutical composition according to claim 11.

22. A method according to claim 19, wherein the cancer is selected from breast cancer, prostate cancer, ovarian cancer, gastric cancer, lung cancer, liver cancer, myeloid cancer and epithelial cancer.

23. The fusion protein of claim 1, further comprising a tetranectin trimerizing domain.

24. An isolated nucleic acid comprising a sequence encoding a fusion protein of claim 1.

25. An isolated nucleic acid according to claim 22, wherein said nucleic acid is selected from the group consisting of SEQ ID NO: 1, SEQ ID NO: 3, SEQ ID NO: 5, SEQ ID NO: 7, SEQ ID NO: 9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 and SEQ ID NO:27.

26. An expression vector comprising the isolated nucleic acid of claim 22.

27. A host cell comprising the expression vector of claim 24.

28. A method for the preparation of a fusion protein as defined in claim 1, said method comprising the steps of (i) expressing the isolated nucleic acid of claim 22 under such conditions that said fusion protein is expressed, and (ii) recovering the fusion protein.
